SEQUENCE LISTING (20 sequences)

SEQ ID NO: 1 - protein, 5 aa, artificial
  CDR1 = hypervariable region 1 of heavy chain of AIN457
NYWMN

SEQ ID NO: 2 - protein, 17 aa, ARTIFICIAL
  CDR2 = hypervariable region 2 of heavy chain of AIN457
AINQDGSEKYYVGSVKG

SEQ ID NO: 3 - protein, 18 aa, ARTIFICIAL
  CDR3 = hypervariable region 3 of heavy chain of AIN457
DYYDILTDYYIHYWYFDL

SEQ ID NO: 4 - protein, 12 aa, ARTIFICIAL
  CDR1′ = hypervariable region 1 of light chain of AIN457
RASQSVSSSYLA

SEQ ID NO: 5 - protein, 7 aa, ARTIFICIAL
  CDR2′ = hypervariable region 2 of light chain AIN457
GASSRAT

SEQ ID NO: 6 - protein, 9 aa, ARTIFICIAL
  CDR3′ = hypervariable region 3 of light chain AIN457
QQYGSSPCT

SEQ ID NO: 7 - DNA, 381 bp, HOMO SAPIENS
  CDS (1)..(381)
gaggtgcagttggtggagtctgggggaggcttggtccagcctggggggtccctgagactctcctgtgcagcctctggattcacctttagtaactattggatgaactgggtccgccaggctccagggaaagggctggagtgggtggccgccataaaccaagatggaagtgagaaatactatgtgggctctgtgaagggccgattcaccatctccagagacaacgccaagaactcactgtatctgcaaatgaacagcctgagagtcgaggacacggctgtgtattactgtgtgagggactattacgatattttgaccgattattacatccactattggtacttcgatctctggggccgtggcaccctggtcactgtctcctca

SEQ ID NO: 8 - protein, 127 aa, HOMO SAPIENS [sequence omitted]

SEQ ID NO: 9 - DNA, 327 bp, HOMO SAPIENS
  CDS (1)..(327)
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtgcaccttcggccaagggacacgactggagattaaacga

SEQ ID NO: 10 - protein, 109 aa, HOMO SAPIENS
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKR

SEQ ID NO: 11 - protein, 10 aa, artificial
  CDR1-x = hypervariable domain x of heavy chain of AIN457
GFTFSNYWMN

SEQ ID NO: 12 - protein, 11 aa, artificial
  CDR2-x = hypervariable domain of heavy chain x of AIN457
AINQDGSEKYY

SEQ ID NO: 13 - protein, 23 aa, ARTIFICIAL
  CDR3-x = hypervariable domain x of heavy chain AIN457
CVRDYYDILTDYYIHYWYFDLWG

SEQ ID NO: 14 - protein, 215 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 457 aa, homo sapiens
EVQLVESGGGLVQPGGSLRLSCAASGFTFSNYWMNWVRQAPGKGLEWVAAINQDGSEKYYVGSVKGRFTISRDNAKNSLYLQMNSLRVEDTAVYYCVRDYYDILTDYYIHYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 7 aa, Homo sapiens
QSVSSSY

SEQ ID NO: 17 - protein, 3 aa, homo sapiens
GAS

SEQ ID NO: 18 - protein, 8 aa, homo sapiens
GFTFSNYW

SEQ ID NO: 19 - protein, 8 aa, homo sapiens
INQDGSEK

SEQ ID NO: 20 - protein, 20 aa, homo sapiens [sequence omitted]

(19) United States
(12) Patent Application Publication
(10) Pub. No.: US 2020/0277369 A1
(43) Pub. Date: Sep. 3, 2020

(54) METHOD OF TREATING HIDRADENTITIS SUPPURATIVA WITH IL-17 ANTAGONISTS

(71) Applicant: NOVARTIS AG, Basel (CH)

(72) Inventors: Christian Loesche, Hegenheim (FR); Anna Maria DE VERA JUAREZ, Basel (CH); Gerard BRUIN, Basel (CH); Farkad Ezzet, Chatham, NJ (US)

(73) Assignee: Novartis AG, Basel (CH)

(21) Appl. No.: 16/761,513

(22) PCT Filed: Nov. 11, 2018
(86) PCT No.: PCT/IB2018/059099

§ 371 Date: May 5, 2020

Related U.S. Application Data
(60) Provisional application No. 62/588,687, filed on Nov. 20, 2017.

(51) Int. Cl. C07K 16/24; A61P 17/00

(52) U.S. Cl. CPC C07K 16/244; A61K 2039/545; A61P 17/00

(57) ABSTRACT

The present disclosure relates to methods for treating Hidradenitis Suppurativa (HS) using IL-17 antagonists, e. g., secukinumab. Also disclosed herein are IL-17 antagonists, e.g., IL-17 antibodies, such as secukinumab, for treating HS patients, as well as medicaments, dosing regimens, pharmaceutical formulations, dosage forms, and kits for use in the disclosed uses and methods.

### RELATED APPLICATIONS

This disclosure claims priority to U.S. Provisional Patent Application No. 62/588,687, filed Nov. 20, 2017, the disclosure of which is incorporated by reference herein in its entirety.

### TECHNICAL FIELD

The present disclosure relates to methods for treating Hidradenitis suppurativa using IL-17 antagonists, e.g., secukinumab.

### BACKGROUND OF THE DISCLOSURE

Hidradenitis suppurativa (HS) (also referred to as acne inversa or Verneuil's disease) is a chronic, recurring, inflammatory disease characterized by deep-seated nodules, sinus tracts, and abscesses that lead to fibrosis in the axillary, inguinal, breast-fold, and anogenital regions. (Revuz and Jemec (2016) Dermatol Clin 34:1-5; Jemec G B. (2012) N Engl J Med 366:158-64). It is associated with substantial pain and comorbidities, including metabolic, psychiatric, and autoimmune disorders, as well as an increased risk of skin cancer. (Revuz (2016); Shlyankevich et al. (2014) J Am Acad Dermatol 71:1144-50; Kohorst et al (2015) J Am Acad Dermatol 73: S27-35; Wolkenstein et al. (2007) J Am Acad Dermatol 56:621-3).

Reported prevalence rates of HS vary from <1% to 4% of the population. (Shlyankevich et al. (2014); Cosmatos et al. (2013) J Am Acad Dermatol 68:412-9; Davis et al. (2015) Skin Appendage Disord 1:65-73; Revuz et al. (2008) J Am Acad Dermatol 59:596-601; McMillan K. (2014) Am J Epidemiol 179:1477-83; Garg et al. (2017) JAMA Dermatol; Jemec et al. (1996) J Am Acad Dermatol 35:191-4). However, the true prevalence is difficult to ascertain because HS is underdiagnosed, and estimates fluctuate with study design, population, and geographic location. (Miller et al. (2016) Dermatol Clin 34:7-16). Although the National Institutes of Health (NIH) does not classify HS as a rare disease, experts generally consider the prevalence of the disease to be <1% of the United States (US) population. (Cosmatos et al. (2013); Genetic and Rare Diseases Information Center. National Institutes of Health. Hidradenitis suppurativa. Available at: //rarediseases.info.nih.gov/diseases/6658/hidradenitis-suppurativa. Accessed Mar. 20, 2017; Gulliver et al. (2016) Rev Endocr Metab Disord 17:343-51).

Current treatment for HS consists of topical and/or systemic antibiotics, hormonal interventions, analgesics and, in selected cases, immunosuppressants, the tumor necrosis factor [TNF] inhibitor monoclonal antibody adalimumab, and surgical excision. (Gulliver et al. (2016); Zouboulis et al. (2015) J Eur Acad Dermatol Venereol 29:619-4414-16; Kimball et al. (2016) N Engl J Med 375:422-34). However, symptom control and lesion resolution are inconsistent among treatments. The recurrence rate is high after discontinuation of antibiotic therapy and long-term treatment with retinoids poses teratogenicity concerns. Moreover, the effectiveness of inflammatory drugs, such as dapsone, fumarates and cyclosporine, is based on small case studies with varying results. As a result of these inconsistent outcomes, and the severity of the HS disease, HS patients utilize healthcare in high-cost settings (e.g., emergency department and inpatient care) more frequently than patients with other chronic inflammatory skin conditions. (Khalsa et al. (2016) J Am Acad Dermatol 73:609-14; Kirby et al. (2014) JAMA Dermatol 150:937-44). Because there is no medical cure for HS, and the disease is physically and psychologically debilitating, there is a clear unmet need to provide safe and effective long-term treatments for HS patients.

### SUMMARY OF THE DISCLOSURE

While the pathogenesis of HS is still not fully understood, Van der Zee et al. (2011) and Kelly et al. (2015) show that IL-17, IL-1β, and TNF-α expression is enhanced in lesional and perilesional skin of HS patients, and Matusiak et al. (2017) show that patients with HS have increased serum levels of IL-17 compared to healthy volunteers, with a tendency toward higher serum concentrations of IL-17 in patients with more advanced disease. (Van der Zee et al. (2011) Br. Ass. Derm. 164:1292-1298 and Kelly et al. (2015) Br. J. Dermatol. 173(6):1431-9; Matusiak et al. (2017) J. Am. Acad. Dermatol. 76(4):670-675). Conversely, Block et al. (2015) found that there were no significant differences in serum concentrations of IL-2R, TNF-α, IL-17A and IL-17F between HS patients and healthy controls, and Banjeree et al. (2017) found no significant difference in proinflammatory cytokines including, e.g., TNF-α, IL-1β, IL-17A, in HS wound effluent versus specimens from chronic wound patients. (Block et al. (2015) Br. J. Dermatol. 174:839-846; Banjeree et al. (2017) Immunological Investigations 46:149-158). Moreover, during an open-label psoriasis study, the IL-17 antagonist, ixekizumab, triggered three separate HS flares in the same patient. (Gordon et al. (2014) J. Am. Acad. Dermatol. 71(6):1176-82; Gordon et al. 72^{nd} Annu. Meet Am Acad Dermatol (AAD) (March 21-25, Denver) 2014, Abst P7617). Thus, whether IL-17 dysregulation is causally linked to HS pathogenesis (i.e., a disease driver) or simply represents an outcome of inflammation and/or wounds caused by other triggers (i.e., a disease passenger) is unclear.

Secukinumab is a recombinant high-affinity, fully human monoclonal anti-human interleukin-17A (IL-17A, IL-17) antibody of the IgG₁/kappa isotype. Secukinumab (see, e.g., WO2006/013107 and WO2007/117749) has a very high affinity for IL-17, i.e., a K_{D} of about 100-200 pM and an IC₅₀ for in vitro neutralization of the biological activity of about 0.67 nM human IL-17A of about 0.4 nM. Thus, secukinumab inhibits antigen at a molar ratio of about 1:1. This high binding affinity makes the secukinumab antibody particularly suitable for therapeutic applications. Furthermore, secukinumab has a long half-life, i.e., about 4 weeks, which allows for prolonged periods between administration, an exceptional property when treating chronic life-long disorders, such as HS.

There are two case reports of secukinumab treatment of HS patients. Schuch et al. (2017) report the treatment of severe recalcitrant HS using secukinumab at a dose of 300 mg SC weekly for 1 month (days 0, 7, 14, 21 and 28), followed by injections at 4-week intervals (Schuch et al. (2017) Acta Derm Venereol. 2018 Jan. 12; 98(1):151-152). Similarly, Thorlacius et al. (2017) report the treatment of an HS patient with Hurley Stage III lesions using 300 mg secukinumab SC weekly for the first four weeks, then 300 mg SC every 4^{th} week. (Thorlacius et al. (2017) Br. J. Dermatol doi: 10.1111/bjd.15769. [Epub ahead of print]). However, in Thorlacius et al. (2017), the patient's reported improvement in the number of boils, pain VAS, and pain/utility/handicap VAS was not well reproduced in the physician reported scores, and the patient's quality of life after treatment initiation did not improve, as reflected in the lack of change in the patient's Dermatology Life Quality Index (DLQI). Moreover, neither Schuch et al. (2017) nor Thorlacius et al. (2017) report whether the treatment used provided sustained responses, or whether response was lost over time.

We have now devised regimens for treating HS patients with secukinumab (and similar IL-17 antagonists, e.g., similar IL-17 antibodies or antigen-binding fragments thereof) that are remarkably effective and provide sustained responses for HS patients. Disclosed herein are methods of treating HS, comprising subcutaneously (SC) administering to a patient in need thereof a dose of about 300 mg-about 450 mg of an IL-17 antibody, or an antigen-binding fragment thereof, weekly during weeks 0, 1, 2, and 3, and thereafter SC at a dose of about 300 mg-about 450 mg:

a) monthly (every 4 weeks), beginning during week 4; or

b) every other week (every 2 weeks), beginning during week 4.

In some embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of human IL-17 comprising Leu74, Tyr85, His86, Met87, Asn88, Va1124, Thr125, Pro126, Ile127, Va1128, His129; b) an IL-17 antibody or antigen-binding fragment thereof that binds to an epitope of human IL-17 comprising Tyr43, Tyr44, Arg46, Ala79, Asp80; c) an IL-17 antibody or antigen-binding fragment thereof that binds to an epitope of an IL-17 homodimer having two mature human IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature human IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; e) an IL-17 antibody that binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days; and f) an IL-17 antibody or antigen-binding fragment thereof comprising: i) an immunoglobulin heavy chain variable domain (V_{H}) comprising the amino acid sequence set forth as SEQ ID NO:8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15. In some embodiments of the disclosed uses, methods and kits, the IL-17 antibody or antigen-binding fragment thereof is a human or humanized antibody. In some embodiments of the disclosed uses, methods and kits, the IL-17 antibody or antigen-binding fragment thereof is secukinumab.

In preferred embodiments, the IL-17 antagonist (e.g., IL-17 antibody or antigen-binding fragment thereof, such as secukinumab) is subcutaneously (SC) administered at a dose of about 300 mg-about 450 mg, e.g., about 300 mg or about 450 mg (e.g., 300 mg or 450 mg). In some embodiments, the IL-17 antagonist (e.g., IL-17 antibody or antigen-binding fragment thereof, such as secukinumab) is administered using an induction regimen, followed by a maintenance regimen. In some embodiments, the induction regimen comprises weekly administration and the maintenance regimen comprises administration every two weeks or every four weeks. In some embodiments, the IL-17 antagonist (e.g., IL-17 antibody or antigen-binding fragment thereof, such as secukinumab) is administered SC at a dose of about 300 mg, e.g., 300 mg, at week 0, 1, 2, 3, and 4 and then every two weeks thereafter. In some embodiments, the IL-17 antagonist (e.g., IL-17 antibody or antigen-binding fragment thereof, such as secukinumab) is administered SC at a dose of about 300 mg, e.g., 300 mg, at week 0, 1, 2, 3, and 4 and then every four weeks thereafter. In some embodiments, the IL-17 antagonist (e.g., IL-17 antibody or antigen-binding fragment thereof, such as secukinumab) is administered SC at a dose of about 450 mg (e.g., 450 mg) at week 0, 1, 2, 3, and 4 and then every four weeks (monthly) thereafter.

### BRIEF DESCRIPTION OF THE FIGURES

FIG. 1 shows HS-PGA responder rate by treatment-Period 1 (PD analysis set 1; n/N) for the CJM112 trial of Example 1. N=number of patients in PD analysis set 1. n=number of HS-PGA responders. An HS-PGA responder in Period 1 is a study participant who had an initial HS-PGA score of at least 3 at baseline (Day 1, inclusion criterion) that decreased by at least 2 points. Subjects who discontinued and did not reach the end of the first treatment period would have been considered as non-responders if the reason for discontinuation was local tolerability failure or adverse event considered by the investigator to be related to test treatment. None of the subjects that discontinued did so due to any of these reasons. A missing post-baseline value resulting from a missing assessment at any given time point up to Week 16 was imputed using the last observation carried forward procedure (LOCF) for the primary efficacy analysis.

FIG. 2 displays the simulated secukinumab PASI 90 responder rates up to week 52 for the different secukinumab regimens in subjects with bodyweight greater than or equal to 90 kg. The curves show median of simulated responder rates, and the surrounding shaded region provides 95% prediction interval of simulations.

FIG. 3A shows two secukinumab dosing regimens with the same loading dose but different maintenance dose, i.e. every two weeks (Q2wks) or every four weeks (Q4wks).

FIG. 3B shows the two proposed secukinumab HS clinical trials, one employing concomitant antibiotics, and the other without concomitant antibiotics.

FIG. 4 shows the predicted secukinumab systemic exposure with 2 and 4 weeks dosing intervals during maintenance at the 300 mg dose level.

FIG. 5 shows simulated PASI 90 responder rates and corresponding trough concentrations (mcg/mL) for secukinumab achieved using 300 mg Q2W and 300 mg Q4W based on patient bodyweight greater or less than 90 kg.

### DETAILED DESCRIPTION OF THE DISCLOSURE

As used herein, IL-17 refers to interleukin-17A (IL-17A).

The term “comprising” encompasses “including” as well as “consisting,” e.g., a composition “comprising” X may consist exclusively of X or may include something additional, e.g., X+Y.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The word “substantially” does not exclude “completely,” e.g., a composition which is “substantially free” from Y may be completely free from Y. Where necessary, the word “substantially” may be omitted from the definition of the disclosure.

The term “antibody” as referred to herein includes naturally-occurring and whole antibodies. A naturally-occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed hypervariable regions or complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system. Exemplary antibodies include secukinumab (Table 1), antibody XAB4 (U.S. Pat. No. 9,193,788), and ixekizumab (U.S. Pat. No. 7,838,638), the disclosures of which are incorporated by reference herein in their entirety.

The term “antigen-binding fragment” of an antibody, as used herein, refers to fragments of an antibody that retain the ability to specifically bind to an antigen (e.g., IL-17). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, CL and CH1 domains; a F(ab)2 fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated CDR. Exemplary antigen-binding fragments include the CDRs of secukinumab as set forth in SEQ ID NOs: 1-6 and 11-13 (Table 1), preferably the heavy chain CDR3. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies are also intended to be encompassed within the term “antibody”. Single chain antibodies and antigen-binding portions are obtained using conventional techniques known to those of skill in the art.

An “isolated antibody”, as used herein, refers to an antibody that is substantially free of other antibodies having different antigenic specificities (e.g., an isolated antibody that specifically binds IL-17 is substantially free of antibodies that specifically bind antigens other than IL-17). The term “monoclonal antibody” or “monoclonal antibody composition” as used herein refer to a preparation of antibody molecules of single molecular composition. The term “human antibody”, as used herein, is intended to include antibodies having variable regions in which both the framework and CDR regions are derived from sequences of human origin. A “human antibody” need not be produced by a human, human tissue or human cell. The human antibodies of the disclosure may include amino acid residues not encoded by human sequences (e.g., mutations introduced by random or site-specific mutagenesis in vitro, by N-nucleotide addition at junctions in vivo during recombination of antibody genes, or by somatic mutation in vivo). In some embodiments of the disclosed processes and compositions, the IL-17 antibody is a human antibody, an isolated antibody, and/or a monoclonal antibody.

The term “IL-17” refers to IL-17A, formerly known as CTLA8, and includes wild-type IL-17A from various species (e.g., human, mouse, and monkey), polymorphic variants of IL-17A, and functional equivalents of IL-17A. Functional equivalents of IL-17A according to the present disclosure preferably have at least about 65%, 75%, 85%, 95%, 96%, 97%, 98%, or even 99% overall sequence identity with a wild-type IL-17A (e.g., human IL-17A), and substantially retain the ability to induce IL-6 production by human dermal fibroblasts.

The term “K_{D}” is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods established in the art. A preferred method for determining the K_{D} of an antibody is by using surface plasmon resonance, or using a biosensor system, e.g., a Biacore® system. In some embodiments, the IL-17 antibody or antigen-binding fragment thereof, e.g., secukinumab, binds human IL-17 with a K_{D} of about 100-250 pM.

The term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity. Standard assays to evaluate the binding affinity of the antibodies toward IL-17 of various species are known in the art, including for example, ELISAs, western blots and RIAs. The binding kinetics (e.g., binding affinity) of the antibodies also can be assessed by assays known in the art, e.g., using a Biacore® analysis.

An antibody that “inhibits” one or more of these IL-17 functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (or when a control antibody of irrelevant specificity is present). An antibody that inhibits IL-17 activity affects a statistically significant decrease, e.g., by at least about 10% of the measured parameter, by at least 50%, 80% or 90%, and in certain embodiments of the disclosed methods and compositions, the IL-17 antibody used may inhibit greater than 95%, 98% or 99% of IL-17 functional activity.

“Inhibit IL-6” as used herein refers to the ability of an IL-17 antibody or antigen-binding fragment thereof (e.g., secukinumab) to decrease IL-6 production from primary human dermal fibroblasts. The production of IL-6 in primary human (dermal) fibroblasts is dependent on IL-17 (Hwang et al., (2004) Arthritis Res Ther; 6:R120-128). In short, human dermal fibroblasts are stimulated with recombinant IL-17 in the presence of various concentrations of an IL-17 binding molecule or human IL-17 receptor with Fc part. The chimeric anti-CD25 antibody Simulect® (basiliximab) may be conveniently used as a negative control. Supernatant is taken after 16 h stimulation and assayed for IL-6 by ELISA. An IL-17 antibody or antigen-binding fragment thereof, e.g., secukinumab, typically has an IC₅₀ for inhibition of IL-6 production (in the presence 1 nM human IL-17) of about 50 nM or less (e.g., from about 0.01 to about 50 nM) when tested as above, i.e., said inhibitory activity being measured on IL-6 production induced by hu-IL-17 in human dermal fibroblasts. In some embodiments of the disclosed methods and compositions, IL-17 antibodies or antigen-binding fragments thereof, e.g., secukinumab, and functional derivatives thereof have an IC₅₀ for inhibition of IL-6 production as defined above of about 20 nM or less, more preferably of about 10 nM or less, more preferably of about 5 nM or less, more preferably of about 2 nM or less, more preferably of about 1 nM or less.

The term “derivative”, unless otherwise indicated, is used to define amino acid sequence variants, and covalent modifications (e.g., pegylation, deamidation, hydroxylation, phosphorylation, methylation, etc.) of an IL-17 antibody or antigen-binding fragment thereof, e.g., secukinumab, according to the present disclosure, e.g., of a specified sequence (e.g., a variable domain). A “functional derivative” includes a molecule having a qualitative biological activity in common with the disclosed IL-17 antibodies. A functional derivative includes fragments and peptide analogs of an IL-17 antibody as disclosed herein. Fragments comprise regions within the sequence of a polypeptide according to the present disclosure, e.g., of a specified sequence. Functional derivatives of the IL-17 antibodies disclosed herein (e.g., functional derivatives of secukinumab) preferably comprise V_{H} and/or V_{L} domains that have at least about 65%, 75%, 85%, 95%, 96%, 97%, 98%, or even 99% overall sequence identity with the V_{H} and/or V_{L} sequences of the IL-17 antibodies and antigen-binding fragments thereof disclosed herein (e.g., the V_{H} and/or V_{L} sequences of Table 1), and substantially retain the ability to bind human IL-17 or, e.g., inhibit IL-6 production of IL-17 induced human dermal fibroblasts.

The phrase “substantially identical” means that the relevant amino acid or nucleotide sequence (e.g., V_{H} or V_{L} domain) will be identical to or have insubstantial differences (e.g., through conserved amino acid substitutions) in comparison to a particular reference sequence. Insubstantial differences include minor amino acid changes, such as 1 or 2 substitutions in a 5 amino acid sequence of a specified region (e.g., V_{H} or V_{L} domain). In the case of antibodies, the second antibody has the same specificity and has at least 50% of the affinity of the same. Sequences substantially identical (e.g., at least about 85% sequence identity) to the sequences disclosed herein are also part of this application. In some embodiments, the sequence identity of a derivative IL-17 antibody (e.g., a derivative of secukinumab, e.g., a secukinumab biosimilar antibody) can be about 90% or greater, e.g., 90%, 91%, 92%, 93%, 94%, 95%, 96%, 97%, 98%, 99% or higher relative to the disclosed sequences.

“Identity” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent identity, and not considering any conservative substitutions as part of the sequence identity. Neither N- or C-terminal extensions nor insertions shall be construed as reducing identity. Methods and computer programs for the alignment are known. The percent identity can be determined by standard alignment algorithms, for example, the Basic Local Alignment Search Tool (BLAST) described by Altshul et al. ((1990) J. Mol. Biol., 215: 403 410); the algorithm of Needleman et al. ((1970) J. Mol. Biol., 48: 444 453); or the algorithm of Meyers et al. ((1988) Comput. Appl. Biosci., 4: 11 17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

“Amino acid(s)” refer to all naturally occurring L-α-amino acids, e.g., and include D-amino acids. The phrase “amino acid sequence variant” refers to molecules with some differences in their amino acid sequences as compared to the sequences according to the present disclosure. Amino acid sequence variants of an antibody according to the present disclosure, e.g., of a specified sequence, still have the ability to bind the human IL-17 or, e.g., inhibit IL-6 production of IL-17 induced human dermal fibroblasts. Amino acid sequence variants include substitutional variants (those that have at least one amino acid residue removed and a different amino acid inserted in its place at the same position in a polypeptide according to the present disclosure), insertional variants (those with one or more amino acids inserted immediately adjacent to an amino acid at a particular position in a polypeptide according to the present disclosure) and deletional variants (those with one or more amino acids removed in a polypeptide according to the present disclosure).

The term “pharmaceutically acceptable” means a nontoxic material that does not interfere with the effectiveness of the biological activity of the active ingredient(s).

The term “administering” in relation to a compound, e.g., an IL-17 binding molecule or another agent, is used to refer to delivery of that compound to a patient by any route.

As used herein, a “therapeutically effective amount” refers to an amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an IL-17 antagonist, e.g., secukinumab) administered alone, the term refers to that ingredient alone. When applied to a combination, the term refers to combined amounts of the active ingredients that result in the therapeutic effect, whether administered in combination, serially or simultaneously.

The term “treatment” or “treat” is herein defined as the application or administration of an IL-17 antibody according to the disclosure, for example, secukinumab or ixekizumab, or a pharmaceutical composition comprising said anti-IL-17 antibody, to a subject or to an isolated tissue or cell line from a subject, where the subject has a particular disease (e.g., HS), a symptom associated with the disease (e.g., HS), or a predisposition towards development of the disease (e.g., HS) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, the phrase “population of patients” is used to mean a group of patients. In some embodiments of the disclosed methods, the IL-17 antagonist (e.g., IL-17 antibody, such as secukinumab) is used to treat a population of HS patients.

As used herein, the phrases “has not been previously treated with a systemic treatment for HS” and “naïve” refer to an HS patient who has not been previously treated with a systemic agent, e.g., methotrexate, cyclosporine, a biological (e.g., ustekinumab, adalimumab or other TNF alpha inhibitors, etc.), etc., for HS. Systemic agents (i.e., agents given orally, by injection, etc.) differ from local agents (e.g., topicals and phototherapy) in that systemic agents have a systemic (whole body) effect when delivered to a patient. In some embodiments of the disclosed methods, regimens, uses, kits, and pharmaceutical compositions, the patient has not been previously administered a systemic treatment for HS.

As used herein, the phrase “has been previously treated with a systemic agent for HS” is used to mean a patient that has previously undergone HS treatment using a systemic agent. Such patients include those previously treated with biologics, such as ustekinumab or TNF-alpha inhibitors, and those previously treated with non-biologics, such as cyclosporine. In some embodiments of the disclosure, the patient has been previously administered a systemic agent for HS. In some embodiments, the patient has been previously administered a systemic agent for HS (e.g., methotrexate, cyclosporine), but the patient has not been previously administered a systemic biological drug (i.e., a drug produced by a living organism, e.g., antibodies, receptor decoys, etc.) for HS (e.g., ustekinumab, ixekizumab, broadalumab, TNF alpha inhibitors (etanercept, adalimumab, remicade, etc.), secukinumab, etc.). In this case, the patient is referred to as “biological-naïve.” In some embodiments, the patient is biological-naïve.

As used herein, the term “TNF failure” refers to a patient who had an inadequate response to or was intolerant to prior treatment with a TNF alpha antagonist (e.g., etanercept, adalimumab, etc.). A patient who has responded adequately to prior treatment with a TNF alpha antagonist (e.g., etanercept, adalimumab, etc.) but has discontinued due to a side effect is termed “intolerant”. TNF failures are also sometimes referred to as “TNF-IR” patients. In some embodiments, prior to administering the IL-17 antagonist, the patient is a TNF failure.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By selecting, selectively treating and selectively administering, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient is selected for treatment based on having HS. In some embodiments, the patient is selected for treatment based on having been diagnosed with HS for at least one year. In some embodiments, the patient is selected for treatment based on having moderate to severe HS. In some embodiments, the patient is selected for treatment based on not having been previously treated with a systemic HS therapy. In some embodiments, the patient is selected for treatment based on having been previously treated with a conventional systemic HS therapy. In some embodiments, the patient is selected for treatment based on having previously had an inadequate response to a conventional systemic HS therapy.

As used herein, “conventional systemic therapy” refers to antibiotics, steroids, retinoids, hormonal therapy, and TNF alpha inhibitors (e.g., etanercept, infliximab, adalimumab, etc.).

### IL-17 Antagonists

The various disclosed processes, kits, uses and methods utilize an IL-17 antagonist, e.g., IL-17 binding molecule (e.g., soluble IL-17 receptor, IL-17 antibody or antigen-binding fragment thereof, e.g., secukinumab) or IL-17 receptor binding molecule (e.g., IL-17 receptor antibody or antigen-binding fragment thereof). In some embodiments, the IL-17 antagonist is an IL-17 binding molecule, preferably an IL-17 antibody or antigen-binding fragment thereof.

In one embodiment, the IL-17 antibody or antigen-binding fragment thereof comprises at least one immunoglobulin heavy chain variable domain (V_{H}) comprising hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3. In one embodiment, the IL-17 antibody or antigen-binding fragment thereof comprises at least one immunoglobulin light chain variable domain (V_{L}′) comprising hypervariable regions CDR1′, CDR2′ and CDR3′, said CDR1′ having the amino acid sequence SEQ ID NO:4, said CDR2′ having the amino acid sequence SEQ ID NO:5 and said CDR3′ having the amino acid sequence SEQ ID NO:6. In one embodiment, the IL-17 antibody or antigen-binding fragment thereof comprises at least one immunoglobulin heavy chain variable domain (V_{H}) comprising hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13.

In one embodiment, the IL-17 antibody or antigen-binding fragment thereof comprises at least one immunoglobulin V_{H} domain and at least one immunoglobulin V_{L} domain, wherein: a) the immunoglobulin V_{H} domain comprises (e.g., in sequence): i) hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; or ii) hypervariable regions CDR1-x, CDR2-x and CDR3-x, said CDR1-x having the amino acid sequence SEQ ID NO:11, said CDR2-x having the amino acid sequence SEQ ID NO:12, and said CDR3-x having the amino acid sequence SEQ ID NO:13; and b) the immunoglobulin V_{L} domain comprises (e.g., in sequence) hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6.

In one embodiment, the IL-17 antibody or antigen-binding fragment thereof comprises: a) an immunoglobulin heavy chain variable domain (V_{H}) comprising the amino acid sequence set forth as SEQ ID NO:8; b) an immunoglobulin light chain variable domain (V_{L}) comprising the amino acid sequence set forth as SEQ ID NO:10; c) an immunoglobulin V_{H} domain comprising the amino acid sequence set forth as SEQ ID NO: 8 and an immunoglobulin V_{L} domain comprising the amino acid sequence set forth as SEQ ID NO:10; d) an immunoglobulin V_{H} domain comprising the hypervariable regions set forth as SEQ ID NO:1, SEQ ID NO:2, and SEQ ID NO:3; e) an immunoglobulin V_{L} domain comprising the hypervariable regions set forth as SEQ ID NO:4, SEQ ID NO:5 and SEQ ID NO:6; f) an immunoglobulin V_{H} domain comprising the hypervariable regions set forth as SEQ ID NO:11, SEQ ID NO:12 and SEQ ID NO:13; g) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or h) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6.

For ease of reference the amino acid sequences of the hypervariable regions of the secukinumab monoclonal antibody, based on the Kabat definition and as determined by the X-ray analysis and using the approach of Chothia and coworkers, is provided in Table 1, below.

TABLE 1
Amino acid sequences of the
hypervariable regions of secukinumab.
Light-Chain

| | | |
|---|---|---|
| CDR1′ | Kabat | R-A-S-Q-S-V-S-S-S-Y-L-A |
| | | (SEQ ID NO: 4) |
| | Chothia | R-A-S-Q-S-V-S-S-S-Y-L-A |
| | | (SEQ ID NO: 4) |
| CDR2′ | Kabat | G-A-S-S-R-A-T |
| | | (SEQ ID NO: 5) |
| | Chothia | G-A-S-S-R-A-T |
| | | (SEQ ID NO: 5) |
| CDR3′ | Kabat | Q-Q-Y-G-S-S-P-C-T |
| | | (SEQ ID NO: 6) |
| | Chothia | Q-Q-Y-G-S-S-P-C-T |
| | | (SEQ ID NO: 6) |

Heavy-Chain

| | | |
|---|---|---|
| CDR1 | Kabat | N-Y-W-M-N |
| | | (SEQ ID NO: 1) |
| CDR1-x | Chothia | G-F-T-F-S-N-Y-W-M-N |
| | | (SEQ ID NO: 11) |
| CDR2 | Kabat | A-I-N-Q-D-G-S-E-K-Y- |
| | | Y-V-G-S-V-K-G (SEQ ID NO: 2) |
| CDR2-x | Chothia | A-I-N-Q-D-G-S-E-K-Y-Y |
| | | (SEQ ID NO: 12) |
| CDR3 | Kabat | D-Y-Y-D-I-L-T-D-Y-Y- |
| | | I-H-Y-W-Y-F-D-L (SEQ ID NO: 3) |
| CDR3-x | Chothia | C-V-R-D-Y-Y-D-I-L-T- |
| | | D-Y-Y-I-H-Y-W-Y-F-D-L-W-G |
| | | (SEQ ID NO: 13) |

In preferred embodiments, constant region domains also comprise suitable human constant region domains, for instance as described in “Sequences of Proteins of Immunological Interest”, Kabat E. A. et al, US Department of Health and Human Services, Public Health Service, National Institute of Health. The DNA encoding the V_{L} of secukinumab is set forth in SEQ ID NO:9. The DNA encoding the V_{H} of secukinumab is set forth in SEQ ID NO:7.

In some embodiments, the IL-17 antibody or antigen-binding fragment thereof (e.g., secukinumab) comprises the three CDRs of SEQ ID NO:10. In other embodiments, the IL-17 antibody or antigen-binding fragment thereof comprises the three CDRs of SEQ ID NO:8. In other embodiments, the IL-17 antibody or antigen-binding fragment thereof comprises the three CDRs of SEQ ID NO:10 and the three CDRs of SEQ ID NO:8. CDRs of SEQ ID NO:8 and SEQ ID NO:10 may be found in Table 1. The free cysteine in the light chain (CysL97) may be seen in SEQ ID NO:6.

In some embodiments, IL-17 antibody or antigen-binding fragment thereof comprises the light chain of SEQ ID NO:14. In other embodiments, the IL-17 antibody or antigen-binding fragment thereof comprises the heavy chain of SEQ ID NO:15. In other embodiments, the IL-17 antibody or antigen-binding fragment thereof comprises the light chain of SEQ ID NO:14 and the heavy domain of SEQ ID NO:15. In some embodiments, the IL-17 antibody or antigen-binding fragment thereof comprises the three CDRs of SEQ ID NO:14. In other embodiments, IL-17 antibody or antigen-binding fragment thereof comprises the three CDRs of SEQ ID NO:15. In other embodiments, the IL-17 antibody or antigen-binding fragment thereof comprises the three CDRs of SEQ ID NO:14 and the three CDRs of SEQ ID NO:15. CDRs of SEQ ID NO:14 and SEQ ID NO:15 may be found in Table 1.

Hypervariable regions may be associated with any kind of framework regions, though preferably are of human origin. Suitable framework regions are described in Kabat E. A. et al, ibid. The preferred heavy chain framework is a human heavy chain framework, for instance that of the secukinumab antibody. It consists in sequence, e.g. of FR1 (amino acid 1 to 30 of SEQ ID NO:8), FR2 (amino acid 36 to 49 of SEQ ID NO:8), FR3 (amino acid 67 to 98 of SEQ ID NO:8) and FR4 (amino acid 117 to 127 of SEQ ID NO:8) regions. Taking into consideration the determined hypervariable regions of secukinumab by X-ray analysis, another preferred heavy chain framework consists in sequence of FR1-x (amino acid 1 to 25 of SEQ ID NO:8), FR2-x (amino acid 36 to 49 of SEQ ID NO:8), FR3-x (amino acid 61 to 95 of SEQ ID NO:8) and FR4 (amino acid 119 to 127 of SEQ ID NO: 8) regions. In a similar manner, the light chain framework consists, in sequence, of FR1′ (amino acid 1 to 23 of SEQ ID NO:10), FR2′ (amino acid 36 to 50 of SEQ ID NO:10), FR3′ (amino acid 58 to 89 of SEQ ID NO:10) and FR4′ (amino acid 99 to 109 of SEQ ID NO:10) regions.

In one embodiment, the IL-17 antibody or antigen-binding fragment thereof (e.g., secukinumab) is selected from a human IL-17 antibody that comprises at least: a) an immunoglobulin heavy chain or fragment thereof which comprises a variable domain comprising, in sequence, the hypervariable regions CDR1, CDR2 and CDR3 and the constant part or fragment thereof of a human heavy chain; said CDR1 having the amino acid sequence SEQ ID NO:1, said CDR2 having the amino acid sequence SEQ ID NO:2, and said CDR3 having the amino acid sequence SEQ ID NO:3; and b) an immunoglobulin light chain or fragment thereof which comprises a variable domain comprising, in sequence, the hypervariable regions CDR1′, CDR2′, and CDR3′ and the constant part or fragment thereof of a human light chain, said CDR1 ‘ having the amino acid sequence SEQ ID NO:4, said CDR2’ having the amino acid sequence SEQ ID NO:5, and said CDR3′ having the amino acid sequence SEQ ID NO:6.

In one embodiment, the IL-17 antibody or antigen-binding fragment thereof is selected from a single chain antibody or antigen-binding fragment thereof that comprises an antigen-binding site comprising: a) a first domain comprising, in sequence, the hypervariable regions CDR1, CDR2 and CDR3, said CDR1 having the amino acid sequence SEQ ID NO:1, said CDR2 having the amino acid sequence SEQ ID NO:2, and said CDR3 having the amino acid sequence SEQ ID NO:3; and b) a second domain comprising, in sequence, the hypervariable regions CDR1′, CDR2′ and CDR3′, said CDR1′ having the amino acid sequence SEQ ID NO:4, said CDR2′ having the amino acid sequence SEQ ID NO:5, and said CDR3′ having the amino acid sequence SEQ ID NO:6; and c) a peptide linker which is bound either to the N-terminal extremity of the first domain and to the C-terminal extremity of the second domain or to the C-terminal extremity of the first domain and to the N-terminal extremity of the second domain.

Alternatively, an IL-17 antibody or antigen-binding fragment thereof as used in the disclosed methods may comprise a derivative of the IL-17 antibodies set forth herein by sequence (e.g., pegylated variants, glycosylation variants, affinity-maturation variants, etc.). Alternatively, the V_{H} or V_{L} domain of an IL-17 antibody or antigen-binding fragment thereof used in the disclosed methods may have V_{H} or V_{L} domains that are substantially identical to the V_{H} or V_{L} domains set forth herein (e.g., those set forth in SEQ ID NO:8 and 10). A human IL-17 antibody disclosed herein may comprise a heavy chain that is substantially identical to that set forth as SEQ ID NO:15 and/or a light chain that is substantially identical to that set forth as SEQ ID NO:14. A human IL-17 antibody disclosed herein may comprise a heavy chain that comprises SEQ ID NO:15 and a light chain that comprises SEQ ID NO:14. A human IL-17 antibody disclosed herein may comprise: a) one heavy chain which comprises a variable domain having an amino acid sequence substantially identical to that shown in SEQ ID NO:8 and the constant part of a human heavy chain; and b) one light chain which comprises a variable domain having an amino acid sequence substantially identical to that shown in SEQ ID NO:10 and the constant part of a human light chain.

Alternatively, an IL-17 antibody or antigen-binding fragment thereof used in the disclosed methods may be an amino acid sequence variant of the reference IL-17 antibodies set forth herein, as long as it contains CysL97. The disclosure also includes IL-17 antibodies or antigen-binding fragments thereof (e.g., secukinumab) in which one or more of the amino acid residues of the V_{H} or V_{L} domain of secukinumab (but not CysL97), typically only a few (e.g., 1-10), are changed; for instance by mutation, e.g., site directed mutagenesis of the corresponding DNA sequences. In all such cases of derivative and variants, the IL-17 antibody or antigen-binding fragment thereof is capable of inhibiting the activity of about 1 nM (=30 ng/ml) human IL-17 at a concentration of about 50 nM or less, about 20 nM or less, about 10 nM or less, about 5 nM or less, about 2 nM or less, or more preferably of about 1 nM or less of said molecule by 50%, said inhibitory activity being measured on IL-6 production induced by hu-IL-17 in human dermal fibroblasts as described in Example 1 of WO 2006/013107.

In some embodiments, the IL-17 antibodies or antigen-binding fragments thereof, e.g., secukinumab, bind to an epitope of mature human IL-17 comprising Leu74, Tyr85, His86, Met87, Asn88, Va1124, Thr125, Pro126, Ile127, Va1128, His129. In some embodiments, the IL-17 antibody, e.g., secukinumab, binds to an epitope of mature human IL-17 comprising Tyr43, Tyr44, Arg46, Ala79, Asp80. In some embodiments, the IL-17 antibody, e.g., secukinumab, binds to an epitope of an IL-17 homodimer having two mature human IL-17 chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain. The residue numbering scheme used to define these epitopes is based on residue one being the first amino acid of the mature protein (i.e., IL-17A lacking the 23 amino acid N-terminal signal peptide and beginning with glycine). The sequence for immature IL-17A is set forth in the Swiss-Prot entry Q16552. In some embodiments, the IL-17 antibody has a K_{D} of about 100-200 pM (e.g., as determined by a Biacore® assay). In some embodiments, the IL-17 antibody has an IC₅₀ of about 0.4 nM for in vitro neutralization of the biological activity of about 0.67 nM human IL-17A. In some embodiments, the absolute bioavailability of subcutaneously (SC) administered IL-17 antibody has a range of about 60%-about 80%, e.g., about 76%. In some embodiments, the IL-17 antibody, such as secukinumab, has an elimination half-life of about 4 weeks (e.g., about 23 to about 35 days, about 23 to about 30 days, e.g., about 30 days). In some embodiments, the IL-17 antibody (such as secukinumab) has a Tₘₐₓ of about 7-8 days.

Particularly preferred IL-17 antibodies or antigen-binding fragments thereof used in the disclosed methods are human antibodies, especially secukinumab as described in Examples 1 and 2 of WO 2006/013107. Other preferred IL-17 antibodies for use in the disclosed methods, kits and regimens are those set forth in U.S. Pat. Nos. 8,057,794; 8,003,099; 8,110,191; and 7,838,638 and US Published Patent Application Nos: 20120034656 and 20110027290, which are incorporated by reference herein in their entirety.

### Methods of Treatment and Uses of IL-17 Antagonists for HS

The disclosed IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof), may be used in vitro, ex vivo, or incorporated into pharmaceutical compositions and administered in vivo to treat HS patients (e.g., human patients).

HS is the chronic, inflammatory, scarring condition involving primarily the intertriginous skin of the axillary, inguinal, inframammary, genito-anal, and perineal areas of the body. It is also referred to as acne inversa. Three diagnostic criteria establish a diagnosis of HS: typical lesions (deep-seated painful nodules [blind] boils in early primary lesions, or abscesses, draining sinuses, bridged scars, and “tombstone” open comedones in secondary lesions); typical topography (axillae, groin, gentials, perineal and perianal regions, buttocks, and infra- and intermammary areas; and chronicity and recurrence (Margesson and Danby (2014) Best Practices and Res. Clin. Ob. And Gyn 28:1013-1027). The physical extent of HS can be classified using Hurley's clinical staging, shown below in Table 2:

TABLE 2
Hurley's Stages of HS. Practically speaking, a patient having Hurley's
stage III may have burned-out Stage III, but active Stage I or II lesions.

| | |
|---|---|
| Stage I | Abscesses only (single or multiple) without sinus tracts and |
| | cicatrization (scarring) |
| Stage II | Abscesses (single or multiple) with tract formation or |
| | cicatrization, single or multiple widely separated lesions |
| | (e.g., >10 cm apart) |
| Stage III | Diffuse or near diffuse involvement, or multiple |
| | interconnecting tracts or abscesses across entire area |

HS consists of follicular plugging, ductal rupture, and secondary inflammation. Patients first experience a plug in the follicular duct, which, over time leads to duct leak and horizontal rupture into the dermis. When repair of the folliculo-pilosebaceous (FPSB) fails, the follicular fragments stimulate three reactions that begin the HS disease course. The first is an inflammatory response, triggered by the innate immune system, causing purulence and tissue destruction, and leading to foreign body reactions and extensive scarring. The second reaction leads to epithelialized sinuses, which may evolve from stem cells derived from the FPSB unit that survive the destruction caused by the inflammatory response. Third, an invasive proliferative gelatinous mass is produced in most cases, consisting of a gel containing inflammatory cells, and, it is postulated, the precursors of the epithelialized elements described above. (See Margesson and Danby (2014)). As used herein, the phrase “slowing HS disease progression” means decelerating the advancement rate of any of the aspects of the HS disease course described above, particularly the inflammatory response. In some embodiments of the disclosure, treatment with the IL-17 antagonist (e.g., secukinumab) slows HS disease progression.

Recurrence of HS in a patient includes the development of papules, pustules or inflammatory nodules, pain and itching, abscesses, draining, and any combination thereof. As used herein, “HS flare” (and the like) is defined as at least a 25% increase in abscesses and inflammatory nodule counts (AN), with a minimum increase of two ANs relative to a baseline. In some embodiments of the disclosure, treatment according to the disclosed methods with the IL-17 antagonist (e.g., secukinumab) prevents HS flares, decreases the severity of HS flares, and/or decreases the frequency of HS flares. In some embodiments, when a population of HS patients is treated according to the disclosed methods, less than 5%, less than 10%, less than 15% or less than 20% experiences a flare during the first 16 weeks of treatment.

As used herein, the phrase “decreasing the severity of HS flares” and the like means reducing the intensity of an HS flare, e.g., reducing the number and/or size of abscesses and/or inflammatory nodules, reducing the strength of a particular flare component (e.g., reducing the number, size, thickness, etc. of abscesses and/or inflammatory nodules, reducing the extent of skin irritation (itching, pain) etc.), and/or reducing the amount of time a flare (or component thereof) persists.

As used herein, the phrase “decreasing the frequency of HS flares” and the like means reducing the incidence of HS flares, e.g., reducing the incidence of abscesses and/or inflammatory nodules. By decreasing the frequency of HS flares, a patient will experience fewer HS relapses. The incidence of flares may be assessed by monitoring a patient over time to determine if the prevalence of flares decreases.

As used herein, the phrase “preventing HS flares” means eliminating future HS flares and/or flare components.

The effectiveness of an HS treatment may be assessed using various known methods and tools that measure HS disease state and/or HS clinical response. Some examples include, e.g., Hurley's staging, a Sartorius score, a modified Sartorius score, the HS physicians' global assessment (HS-PGA) score, a visual analog scale (VAS) or numeric rating scale (NRS) to rate skin related pain, the dermatology life quality index (DLQI), HS clinical response based on sum of abscesses and inflammatory nodules (HiSCR), simplified HiSCR, EuroQuol-5D (EQSD), hospital anxiety and depression scale, healthcare resources utilization, Hidradenitis Suppurativa Severity Index (HSSI), Work productivity index (WPI), inflamed body surface area (BSA), Acne Inversa Severity Index (AISI) etc. (see, e.g., Deckers and Prens (2016) Drugs 76:215-229; Sartorius et al. (2009) Br. J. Dermatol 161:831-39; Chiricozzi et al. (2015) Wounds 27(10):258-264). In some embodiments, an HS patient achieves a HiSCR in response to HS treatment. In some embodiments, when a population of HS patients is treated according to the disclosed methods, at least 41%, at least 50%, at least 51%, at least 61%, or at least 71% achieve a HiSCR by week 16 of treatment.

Preferred scoring systems for treatment response are the HiSCR, simplified HiSCR, NRS (especially NRS30), modified Sartorius score, HS-PGA, inflammatory lesion count (count of abscesses, inflammatory nodules, and/or draining fistulae), and the DLQI.

The Hidradenitis Suppurativa Clinical Response (HiSCR) is a measure of clinical response to HS treatment. A HiSCR response to treatment (compared to baseline) is as follows: 1) at least 50% reduction in abscesses and inflammatory nodules, and 2) no increase in the number of abscesses, and 3) no increase in the number of draining fistulae. As used herein the “simplified HiSCR” or “sHiSCR” refers to a modified HiSCR that does not include the abscess count versus baseline (item #2, above) when assessing progression of lesions. In preferred embodiments, an HS patient achieves a simplified HiSCR in response to HS treatment. In some embodiments, when a population of HS patients is treated according to the disclosed methods, at least 41%, at least 50%, at least 51%, at least 61%, or at least 71% achieve a simplified HiSCR by week 16 of treatment.

Pain can be assessed using a numeric rating scale (NRS). In some embodiments, an HS patient achieves an improved NRS in response to HS treatment. NRS30 is defined as at least 30% reduction in pain and at least 1 unit reduction from baseline in Patient's Global Assessment (PGA) of Skin Pain from baseline in patients with a baselines score of 3 or higher. In some embodiments, an HS patient achieves an NRS30 in response to HS treatment. In some embodiments, when a population of HS patients is treated according to the disclosed methods, at least 30%, at least 40%, at least 50%, or at least 60% achieve an NRS30 by week 16 of treatment.

The DLQI is the most established dermatological life quality instrument. It consists of questions regarding the impact of the skin disease on feelings and different aspects of daily life activities during the last week. Each question is scored from 0 (not at all) to 3 (very much). A total of 30 points is the maximum score, where 0-1 is regarded as no effect, 2-5 small, 6-10 moderate, 11-20 very large and 21-30 as extremely large effect on the patient's life. (See Finlay and Khan (1994) Clin Exp Dermatol 19:210-16). In some embodiments, an HS patient achieves an improved DLQI in response to HS treatment.

The Sartorius HS score (also called the HS score, or HSS) is made by counting involved regions, nodules, and sinus tracts in an HS patient. (Sartorius et al. (2003) Br J Dermatol 149:211-13). The modified Sartorius HS score is a revision of the original version of the HSS by making minor simplifications which made it more practical to use, e.g., fewer specific lesions to include in the score, changes to the number of points given for each parameter, etc. (Sartorius et al. (2009) Br. J Dermatol. 161:831-839). In some embodiments, an HS patient achieves an improved modified Sartorius HS in response to HS treatment.

The HS physicians' global assessment (HS-PGA) is a 6-scale evaluating scale (scores range from 0-5) based on the number of HS lesions (i.e., abscesses, draining fistulas, inflammatory nodules, and noninflammatory nodules). (Chiricozzi et al. (2015) Wounds 27(10):258-264). In some embodiments, an HS patient achieves an improved HS-PGA in response to HS treatment. In some embodiments, an HS patient achieves an HS-PGA score of clear, minimal or mild, with at least a 2-grade improvement from baseline in response to HS treatment.

In some embodiments, the patient is treated for HS according to the claimed methods for at least 36 weeks, at least 48 weeks, at least 52 weeks, or at least 2 years. In some embodiments, when a population of HS patients are treated according to the disclosed methods, at least 30%, at least 40%, at least 50%, at least 60%, at least 70%, at least 80% or at least 90% of patients who have responded to treatment by week 16 (e.g., patients achieving a HiSCR or simplified HiSCR by week 16) have sustained response after 1 year (52 weeks) of treatment. As used herein the term “sustained” means that an outcome or goal (e.g., pain reduction, inflammation reduction) is substantially maintained for a given time.

As used herein, the phrase “moderate to severe” refers to HS disease in which patients have ≥5 active, inflammatory lesions [i.e., abscesses and/or inflammatory nodules], affecting at least 2 distinct anatomical areas. In some embodiments, the HS patient has moderate to severe HS disease.

In some embodiments, the patient has been diagnosed with HS for at least one year.

In some embodiments, the patient does not have extensive scarring as a result of HS (i.e., <20 fistulas, draining or not draining).

In some embodiments the patient previously had an inadequate response to conventional systemic HS therapy.

In some embodiments, the patient is an adolescent patient (≥12 years of age) having moderate to severe HS. In some embodiments, the patient is an adult patient having moderate to severe HS.

In some embodiments, in response to treatment according to the claimed methods, the patient experiences rapid reduction in pain, as measured by VAS or NRS, as early as 1 week after initial dosing.

In some embodiments, the patient is a candidate for systemic therapy, i.e., the HS disease is sufficiently severe (e.g., >5% BSA, Hurley stage II or II, etc.) to require systemic intervention.

In some embodiments, the patient is an adult human patient having HS. Is some embodiments, the patient is a pediatric human patient having HS. The upper age limit used to define a pediatric patient varies among experts, and can include adolescents up to the age of 21 (see, e.g., Berhman R E, Kliegman R, Arvin A M, Nelson W E. Nelson Textbook of Pediatrics, 15th Ed. Philadelphia: W.B. Saunders Company; 1996; 2. Rudolph A M, et al. Rudolph's Pediatrics, 21st Ed. New York: McGraw-Hill; 2002; and Avery M D, First L R. Pediatric Medicine, 2nd Ed. Baltimore: Williams & Wilkins; 1994). As used herein, the term “Pediatric” generally refers to a human who is ≥sixteen years, which is the definition of a pediatric human used by the US FDA. Other examples of pediatric patients, however, include those ≥14 years of age and ≥12 years of age.

In some embodiments, the pediatric patient is administered a SC dose of the IL-17 antibody (e.g., secukinumab) weekly during week 0, 1, 2, 3, and 4, and then every two weeks thereafter as a dose of about 300 mg, regardless of the patient's weight.

In some embodiments, the pediatric patient is administered a SC dose of the IL-17 antibody (e.g., secukinumab) weekly during week 0, 1, 2, 3, and 4, and then every four weeks (monthly) thereafter as a dose of about 300 mg, regardless of the patient's weight.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs <25 kg or 150 mg if the patient weighs >25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 75 mg if the patient weighs <50 kg or 150 mg if the patient weighs >50 kg.

In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs <25 kg or 300 mg if the patient weighs >25 kg. In some embodiments, the pediatric patient is administered a SC dose of the IL-17 antibody (e.g., secukinumab) weekly during week 0, 1, 2, 3, and 4, and then every two weeks or every four weeks thereafter as a dose of about 150 mg if the patient weighs <50 kg or 300 mg if the patient weighs >50 kg.

In some embodiments, in response to treatment according to the claimed methods, the patient experiences rapid reduction in CRP, as measured by standard CRP assay or a high sensitivity CRP (hsCRP) assay, as early as 1 week after initial dosing. As used herein, “C-reactive protein” and “CRP” refer to serum C-reactive protein, a plasma protein commonly used as an indicator of the acute phase response to inflammation. The level of CRP in plasma may be given in any concentration, e.g., mg/dl, nmol/L. Levels of CRP may be measured by a variety of standard assays, e.g., radial immunodiffusion, electroimmunoassay, immunoturbidimetry, ELISA, turbidimetric methods, fluorescence polarization immunoassay, and laser nephelometry. Testing for CRP may employ a standard CRP test or a high sensitivity CRP (hs-CRP) test (i.e., a high sensitivity test that is capable of measuring low levels of CRP in a sample using laser nephelometry). Kits for detecting levels of CRP may be purchased from various companies, e.g., Calbiotech, Inc, Cayman Chemical, Roche Diagnostics Corporation, Abazyme, DADE Behring, Abnova Corporation, Aniara Corporation, Bio-Quant Inc., Siemens Healthcare Diagnostics, etc.

The IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof), may be used as a pharmaceutical composition when combined with a pharmaceutically acceptable carrier. Such a composition may contain, in addition to an IL-17 antagonist, carriers, various diluents, fillers, salts, buffers, stabilizers, solubilizers, and other materials known in the art. The characteristics of the carrier will depend on the route of administration. The pharmaceutical compositions for use in the disclosed methods may also contain additional therapeutic agents for treatment of the particular targeted disorder. For example, a pharmaceutical composition may also include anti-inflammatory agents. Such additional factors and/or agents may be included in the pharmaceutical composition to produce a synergistic effect with the IL-17 binding molecules, or to minimize side effects caused by the IL-17 antagonists, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof). In preferred embodiments, the pharmaceutical compositions for use in the disclosed methods comprise secukinumab at 150 mg/ml.

Pharmaceutical compositions for use in the disclosed methods may be manufactured in conventional manner. In one embodiment, the pharmaceutical composition is provided in lyophilized form. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinized blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution. Other formulations comprise ready-to-use liquid formulations.

Antibodies, e.g., antibodies to IL-17, are typically formulated either in ready-to-use aqueous forms for parenteral administration or as lyophilisates for reconstitution with a suitable diluent prior to administration. In preferred embodiments of the disclosed methods and uses, the IL-17 antagonist, e.g., IL-17 antibody, e.g., secukinumab, is formulated as ready-to-use (i.e., a stable ready-to-use) liquid pharmaceutical formulation. In some embodiments of the disclosed methods and uses, the IL-17 antagonist, e.g., IL-17 antibody, e.g., secukinumab, is formulated as a lyophilisate. Suitable lyophilisate formulations can be reconstituted in a small liquid volume (e.g., 2 mL or less, e.g., 2 mL, 1 mL, etc.) to allow subcutaneous administration and can provide solutions with low levels of antibody aggregation. The use of antibodies as the active ingredient of pharmaceuticals is now widespread, including the products HERCEPTIN™ (trastuzumab), RITUXAN™ (rituximab), SYNAGIS™ (palivizumab), etc. Techniques for purification of antibodies to a pharmaceutical grade are known in the art. When a therapeutically effective amount of an IL-17 antagonist, e.g., IL-17 binding molecules (e.g., IL-17 antibody or antigen-binding fragment thereof, e.g., secukinumab) or IL-17 receptor binding molecules (e.g., IL-17 antibody or antigen-binding fragment thereof) is administered by intravenous, cutaneous or subcutaneous injection, the IL-17 antagonist will be in the form of a pyrogen-free, parenterally acceptable solution. A pharmaceutical composition for intravenous, cutaneous, or subcutaneous injection may contain, in addition to the IL-17 antagonist, an isotonic vehicle such as sodium chloride, Ringer's solution, dextrose, dextrose and sodium chloride, lactated Ringer's solution, or other vehicle as known in the art.

In practicing some of the methods of treatment or uses of the present disclosure, a therapeutically effective amount of an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) is administered to a patient, e.g., a mammal (e.g., a human). While it is understood that the disclosed methods provide for treatment of HS patients using an IL-17 antagonist (e.g., secukinumab), this does not preclude that, if the patient is to be ultimately treated with an IL-17 antagonist, such IL-17 antagonist therapy is necessarily a monotherapy. Indeed, if a patient is selected for treatment with an IL-17 antagonist, then the IL-17 antagonist (e.g., secukinumab) may be administered in accordance with the methods of the disclosure either alone or in combination with other agents and therapies for treating HS patients, e.g., in combination with at least one additional HS agent. When co-administered with one or more additional HS agent(s), an IL-17 antagonist may be administered either simultaneously with the other agent, or sequentially. If administered sequentially, the attending physician will decide on the appropriate sequence of administering the IL-17 antagonist in combination with other agents and the appropriate dosages for co-delivery.

Various therapies may be beneficially combined with the disclosed IL-17 antibodies, such as secukinumab, during treatment of HS. Such conventional therapies include topical treatments (creams [non-steroidal or steroidal], washes, antiseptics,), systemic treatments (e.g., with biologicals, antibiotics, hormones, retinoids, or chemical entities), antiseptics, photodynamic therapy, and surgical intervention (laser, draining or incision, excision). Additional combination therapies include use of JAK inhibitors, IL-23 targeted treatments (e.g., guselkumab), microbiome treatment, and sclerotherapy.

Non-limiting examples of topical HS agents for use with the disclosed IL-17 antibodies, such as secukinumab, include benzoyl peroxide, topical steroid creams, topical antibiotics in the aminoglycoside group, such as clindamycin, gentamicin, and erythromycin, resorcinol cream, iodine scrubs, and chlorhexidine.

Non-limiting examples of HS agents used in systemic treatment for use with the disclosed IL-17 antibodies, such as secukinumab, include further IL-17 antagonists (ixekizumab, brodalumab, CJM112), tumor necrosis factor-alpha (TNF-alpha) blockers (such as Enbrel® (etanercept), Humira® (adalimumab), Remicade® (infliximab) and Simponi® (golimumab)), interleukin 12/23 blockers (such as Stelara® (ustekinumab), tasocitinib, and briakinumab), p19 inhibitors, PDE4 inhibitors, leukotriene A4Hydrolase inhibitors, complement pathway inhibitors, C5a inhibitors, IL-1 antagonists (canakinumab, rilonacept, anakinra), CXCR1/2 inhibitors, IL-18 antagonists, IL-6 antagonists, CD20 antagonists, CTLA4 antagonists, IL-8 antagonists, B-cell depletors (particularly CD20 antagonists, as well as BAFF-R and CD40 antagonists), IL-21 antagonists, IL-22 antagonist, VEGF antagonists, CXCL antagonists, MMP antagonists, and defensin antagonists (e.g., receptor decoys, antagonistic antibodies, etc.).

Additional HS agents for use in combination with the disclosed IL-17 antibodies, such as secukinumab, during treatment of HS include retinoids, such as Acitretin (e.g., Soriatane®) and isotretinoin, immune system suppressants (e.g., rapamycin, T-cell blockers [e.g., Amevive® (alefacept) and Raptiva® [efalizumab]) cyclosporine, methotrexate, mycophenolate mofetil, mycophenolic acid, leflunomide, tacrolimus, etc.), hydroxyurea (e.g., Hydrea®), sulfasalazine, 6-thioguanine, fumarates (e.g, dimethylfumarate and fumaric acid esters), azathioprine, colchicine, alitretinoin, steroids, corticosteroids, certolizumab, apremilast, mometasone, rosiglitazone, pioglitazone, botulinum toxin, triamcinolone, IFX-1 (InflaRx), bimekizumab (UCB), MaBp1 (XBiotech), LY-3041658 (Eli Lilly), 1E-2232 (Immunwork), NSAIDs, prescription narcotics, ketoprofen, codeine, gabapentin, pregabalin gentanyl, antibiotics (topical, oral, IV) (e.g., clindamycin, rifampin, tetracycline, sarecycline, doxycycline, minocycline, lymecycline, trimethoprim-sulfamethoxazole, erythromycin, ceftriaxone, moxifloxacin, metronidazole, separately or as combinations), corticosteroid (injectable or oral), antiandrogen/hormonal therapy (oral contraceptives, spironolactone, finasteride, dutasteride, progesterone IUD, cyproterone acetate, ethinyloestradiol, gestodene, norgestimate, desogestrel, drospirenone, spironolactone), Triamcinolone Acetonide, MEDI8968, hydroxychloroquine, dapsone, metformin, adapalene, azelaic acid and zinc.

Preferred combinations for used in the disclosed kits, methods, and uses include the IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) in combination with a TNF alpha inhibitor (e.g., adalimumab) or an IL-1β blocker (e.g., canakinumab).

A skilled artisan will be able to discern the appropriate dosages of the above HS agents for co-delivery with the disclosed IL-17 antibodies, such as secukinumab.

An IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) is conveniently administered parenterally, e.g., intravenously (e.g., into the antecubital or other peripheral vein), intramuscularly, or subcutaneously. The duration of intravenous (IV) therapy using a pharmaceutical composition of the present disclosure will vary, depending on the severity of the disease being treated and the condition and personal response of each individual patient. Also contemplated is subcutaneous (SC) therapy using a pharmaceutical composition of the present disclosure. The health care provider will decide on the appropriate duration of IV or SC therapy and the timing of administration of the therapy, using the pharmaceutical composition of the present disclosure. In preferred embodiments, the IL-17 antagonist (e.g., secukinumab) is administered via the subcutaneous (SC) route.

The IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient intravenously (IV), e.g., at about 10 mg/kg every other week during week 0, 2, and 4 and thereafter administered to the patient subcutaneously (SC), e.g., at about 300 mg-about 450 mg (e.g., about 300 mg, about 450 mg) every two weeks, beginning during week 6. In this manner, the patient may be dosed IV with about 10 mg/kg during week 0, 2, 4, and then the patient is dosed SC with about 300 mg-about 450 mg (e.g., about 300 mg, about 450 mg) of the IL-17 antagonist (e.g., secukinumab) during week 6, 8, 10, 12, 14, etc.

Alternatively IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient intravenously (IV), e.g., at about 10 mg/kg every other week during week 0, 2, and 4 and thereafter administered to the patient subcutaneously (SC), e.g., at about 300 mg-about 450 mg (e.g., about 300 mg, about 450 mg) every month (every 4 weeks), beginning during week 8. In this manner, the patient may be dosed IV with about 10 mg/kg during week 0, 2, 4, and then the patient is dosed SC with about 300 mg-about 450 mg (e.g., about 300 mg, about 450 mg) of the IL-17 antagonist (e.g., secukinumab) during week 8, 12, 16, 20, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC, e.g., at about 300 mg-about 450 mg (e.g., about 300 mg, about 450 mg) weekly during weeks 0, 1, 2, and 3, and thereafter administered to the patient SC, e.g., at about 300 mg-about 450 mg (e.g., about 300 mg, about 450 mg) every two weeks, beginning during week 4. In this manner, the patient is dosed SC with about 300 mg-about 450 mg (e.g., about 300 mg, about 450 mg) of the IL-17 antagonist (e.g., secukinumab) during weeks 0, 1, 2, 3, 4, 6, 8, 10, 12, etc.

Preferably, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC, e.g., at about 300 mg-about 450 mg (e.g., about 300 mg, about 450 mg) weekly during weeks 0, 1, 2, and 3, and thereafter administered to the patient SC, e.g., at about 300 mg-about 450 mg (e.g., about 300 mg, about 450 mg) monthly (every 4 weeks), beginning during week 4. In this manner, the patient is dosed SC with about 300 mg-about 450 mg (e.g., about 300 mg, about 450 mg) of the IL-17 antagonist (e.g., secukinumab) during weeks 0, 1, 2, 3, 4, 8, 12, 16, 20, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient SC at a dose sufficient to provide a trough concentration above 30 mcg/mL, above 40 mgc/mL, above 60 mcg/mL, above 80 mcg/mL, or above 100 mcg/mL during the maintenance regimen.

More preferably, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC at about 300 mg-about 450 mg (e.g., about 300 mg, about 450 mg) every two weeks. In this manner, the patient is dosed SC with about 300 mg-about 450 mg (e.g., about 300 mg, about 450 mg) of the IL-17 antagonist (e.g., secukinumab) during weeks 0, 2, 4, 6, 8, 12, etc.

Alternatively, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient without a loading regimen, e.g., the antagonist may be administered to the patient SC at about 300 mg-about 450 mg (e.g., about 300 mg, about 450 mg) every four weeks. In this manner, the patient is dosed SC with about 300 mg-about 450 mg (e.g., about 300 mg, about 450 mg) of the IL-17 antagonist (e.g., secukinumab) during weeks 0, 4, 8, 12, 16, 20, etc.

Alternatively, the IL-17 antagonists, e.g., IL-17 antibodies, e.g., secukinumab, can also be delivered locally using intralesional injections, as well as orally (e.g., into the intestinal lumen using Rani Therapeutics technology, e.g., technology set forth in U.S. Pat. Nos. 8,734,429; 9,492,378; 9,456,988; 9,415,004; 9,6297,99; 9,757,548; 9,757,514; 9,402,806; US Pub. Appln. 2017/0189659, 2017/0100459)

It will be understood that dose escalation may be required for certain patients, e.g., HS patients that display inadequate response (e.g., as measured by any of the HS scoring systems disclosed herein, e.g., HiSCR, simplified HiSCR, NRS [especially NRS30], modified Sartorius score, HS-PGA, inflammatory lesion count (count of abscesses, inflammatory nodules, and/or draining fistulae), DLQI, etc.) to treatment with the IL-17 antagonists, e.g., IL-17 binding molecules (e.g., IL-17 antibody or antigen-binding fragment thereof, e.g., secukinumab) or IL-17 receptor binding molecules (e.g., IL-17 receptor antibody or antigen-binding fragment thereof) by week 12, week 16, week 20, week 24, week 48 or week 52 of treatment. Thus, SC dosages of secukinumab may be greater than about 300 mg-about 450 mg SC, e.g., about 350 mg, about 400 mg, about 450 mg (in the case of an original 300 mg dose); about 500 mg, about 550 mg, about 600 mg (in the case of an original 450 mg dose), etc.; similarly, IV dosages may be greater than about 10 mg/kg, e.g., about 11 mg/kg, 12 mg/kg, 15 mg/kg, 20 mg/kg, 25 mg/kg, 30 mg/kg, 35 mg/kg, etc. It will also be understood that dose reduction may also be required for certain patients, e.g., HS patients that display adverse events or an adverse response to treatment with the IL-17 antagonist (e.g., IL-17 antibody or antigen-binding fragment thereof, e.g., secukinumab). Thus, dosages of the IL-17 antagonist (e.g., IL-17 antibody or antigen-binding fragment thereof, e.g., secukinumab), may be less than about 300 mg-about 450 mg SC, e.g., about 250 mg, about 200 mg, about 150 mg (in the case of an original 300 mg dose); about 400 mg, about 350 mg, about 300 mg (in the case of an original 450 mg dose), etc. Similarly, IV dosages may be less than about 10 mg/kg, e.g., about 9 mg/kg, 8 mg/kg, 5 mg/kg, 4 mg/kg, 3 mg/kg, 2 mg/kg, 1 mg/kg, etc. In some embodiments, the IL-17 antagonist, e.g., IL-17 binding molecule (e.g., IL-17 antibody or antigen-binding fragment thereof, e.g., secukinumab) or IL-17 receptor binding molecule (e.g., IL-17 receptor antibody or antigen-binding fragment thereof) may be administered to the patient at an initial dose of 300 mg or 450 mg delivered SC, and the dose is then escalated to about 450 mg (in the case of an original 300 mg dose) or about 600 mg (in the case of an original 450 mg dose) if needed, as determined by a physician.

As used herein, “fixed dose” refers to a flat dose, i.e., a dose that is unchanged regardless of a patient's characteristics. Thus, a fixed dose differs from a variable dose, such as a body-surface area-based dose or a weight-based dose (typically given as mg/kg). In preferred embodiments of the disclosed methods, uses, pharmaceutical compositions, kits, etc., the HS patient is administered fixed doses of the IL-17 antibody, e.g., fixed doses of secukinumab, e.g., fixed doses of about 75 mg-about 450 mg secukinumab, e.g., about 75 mg, about 150 mg, about 300 mg, about 400 mg or about 450 mg secukinumab.

The timing of dosing is generally measured from the day of the first dose of secukinumab (which is also known as “baseline”). However, health care providers often use different naming conventions to identify dosing schedules, as shown in Table 3.

TABLE 3
Common naming conventions for dosing regimens. Bolded
items refer to the naming convention used herein.

| | Week | | | | | | | | | | | |
|---|---|---|---|---|---|---|---|---|---|---|---|---|
| | 0/1 | 1/2 | 2/3 | 3/4 | 4/5 | 5/6 | 6/7 | 7/8 | 8/9 | 9/10 | 10/11 | etc |
| 1^{st} day | 0/1 | 7/8 | 14/15 | 21/22 | 28/29 | 35/36 | 42/43 | 49/50 | 56/57 | 63/64 | 70/71 | etc. |
| of week | | | | | | | | | | | | |

Notably, week zero may be referred to as week one by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the IL-17 antibody regardless of whether the physician refers to a particular week as “week 1” or “week 2”.

In a one dosing regimen, the antibody is administered during week 0, 1, 2, 3, 4, 8, 12, 16, 20, etc. Some providers may refer to this regimen as weekly for five weeks and then monthly (or every 4 weeks) thereafter, beginning during week 8, while others may refer to this regimen as weekly for four weeks and then monthly (or every 4 weeks) thereafter, beginning during week 4. It will be appreciated by a skilled artisan that administering a patient an injection at weeks 0, 1, 2 and 3, followed by once monthly dosing starting at week 4 is the same as: 1) administering the patient an injection at weeks 0, 1, 2, 3, and 4, followed by once monthly dosing starting at week 8; 2) administering the patient an injection at weeks 0, 1, 2, 3 and 4 followed by dosing every 4 weeks; and 3) administering the patient an injection at weeks 0, 1, 2, 3 and 4 followed by monthly administration.

In one dosing regimen, the antibody is administered during week 0, 1, 2, 3, 4, 6, 8, 10, 12, etc. Some providers may refer to this regimen as weekly for five weeks and then every other week (or every 2 weeks) thereafter, beginning during week 6, while others may refer to this regimen as weekly for four weeks and then every other week (or every 2 weeks) thereafter, beginning during week 4. It will be appreciated by a skilled artisan that administering a patient an injection at weeks 0, 1, 2 and 3, followed by administration every other week (or every 2 weeks) starting at week 4 is the same as: 1) administering the patient an injection at weeks 0, 1, 2, 3, and 4, followed by dosing every other week (or every 2 weeks) starting at week 6; 2) administering the patient an injection at weeks 0, 1, 2, 3 and 4 followed by dosing every 2 weeks; and 3) administering the patient an injection at weeks 0, 1, 2, 3 and 4 followed by every other week administration.

As used herein, the phrase “formulated at a dosage to allow [route of administration] delivery of [a designated dose]” is used to mean that a given pharmaceutical composition can be used to provide a desired dose of an IL-17 antagonist, e.g., an IL-17 antibody, e.g., secukinumab, via a designated route of administration (e.g., SC or IV). As an example, if a desired SC dose is 300 mg, then a clinician may use 2 ml of an IL-17 antibody formulation having a concentration of 150 mg/ml, 1 ml of an IL-17 antibody formulation having a concentration of 300 mg/ml, 0.5 ml of an IL-17 antibody formulation having a concentration of 600 mg/ml, etc. In each such case, these IL-17 antibody formulations are at a concentration high enough to allow subcutaneous delivery of the IL-17 antibody. Subcutaneous delivery typically requires delivery of volumes of less than or equal to about 2 ml, preferably a volume of about 1 ml or less. Preferred formulations are ready-to-use liquid pharmaceutical compositions comprising about 25 mg/mL to about 150 mg/mL secukinumab, about 10 mM to about 30 mM histidine pH 5.8, about 200 mM to about 225 mM trehalose, about 0.02% polysorbate 80, and about 2.5 mM to about 20 mM methionine.

As used herein, the phrase “container having a sufficient amount of the IL-17 antagonist to allow delivery of [a designated dose]” is used to mean that a given container (e.g., vial, pen, syringe) has disposed therein a volume of an IL-17 antagonist (e.g., as part of a pharmaceutical composition) that can be used to provide a desired dose. As an example, if a desired dose is 300 mg, then a clinician may use 2 mL from a container that contains an IL-17 antibody formulation with a concentration of 150 mg/mL, 1 mL from a container that contains an IL-17 antibody formulation with a concentration of 300 mg/mL, 0.5 mL from a container contains an IL-17 antibody formulation with a concentration of 600 mg/ml, etc. In each such case, these containers have a sufficient amount of the IL-17 antagonist to allow delivery of the desired 300 mg dose.

In some embodiments of the disclosed uses, methods, and kits, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within two pre-filled syringes, injection pens, or autoinjectors, each having 1 ml of the pharmaceutical formulation. In this case, the patient receives two injections of 1 ml each, for a total dose of 300 mg, during each administration. In some embodiments, the dose of the IL-17 antibody (e.g., secukinumab) or an antigen-binding fragment thereof is about 300 mg, the IL-17 antibody (e.g., secukinumab) or an antigen-binding fragment thereof is comprised in a liquid pharmaceutical formulation at a concentration of 150 mg/ml, and 2 ml of the pharmaceutical formulation is disposed within an autoinjector or PFS. In this case, the patient receives one injection of 2 ml, for a total dose of 300 mg, during each administration. In methods employing one injection of 2 ml (e.g., via a single PFS or autoinjector) (i.e., a “single-dose preparation”), the drug exposure (AUC) and maximal concentration (Cₘₐₓ) is equivalent (similar to, i.e., within the range of acceptable variation according to US FDA standards) to methods employing two injections of 1 ml (e.g., via two PFSs or two AIs) (i.e., a “multiple-dose preparation”).

Disclosed herein are methods of treating hidradenitis suppurativa (HS), comprising subcutaneously (SC) administering to a patient in need thereof a dose of about 300 mg-about 450 mg of an IL-17 antibody (e.g., secukinumab) or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4. Also disclosed herein is an IL-17 antibody (e.g. secukinumab) or an antigen-binding fragment thereof, for use in treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4. Alternatively, disclosed herein is an IL-17 antibody (e.g. secukinumab) or an antigen-binding fragment thereof, for use in the manufacture of a medicament for treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4.

Disclosed herein are methods of treating hidradenitis suppurativa (HS), comprising subcutaneously (SC) administering to a patient in need thereof a dose of about 300 mg-about 450 mg of an IL-17 antibody (e.g., secukinumab) or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days. Also disclosed herein is an IL-17 antibody (e.g. secukinumab) or an antigen-binding fragment thereof, for use in treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE®), and wherein the IL-17 antibody has an in vivo half-life of about 23 to about 30 days. Alternatively, disclosed herein is an IL-17 antibody (e.g. secukinumab) or an antigen-binding fragment thereof, for use in the manufacture of a medicament for treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or an antigen-binding fragment thereof binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system (e.g., BIACORE), and wherein the IL-17 antibody has an in vivo half-life of about 23 to about 30 days.

Disclosed herein are methods of treating hidradenitis suppurativa (HS), comprising subcutaneously (SC) administering to a patient in need thereof a dose of about 300 mg-about 450 mg of an IL-17 antibody (e.g., secukinumab) or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or antigen-binding fragment thereof comprises: i) an immunoglobulin V_{H} domain comprising the amino acid sequence set forth as SEQ ID NO:8 and an immunoglobulin V_{L} domain comprising the amino acid sequence set forth as SEQ ID NO:10; ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO: 5 and SEQ ID NO:6; or iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO: 5 and SEQ ID NO:6. Also disclosed herein is an IL-17 antibody (e.g. secukinumab) or an antigen-binding fragment thereof, for use in treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or an antigen-binding fragment thereof comprises: i) an immunoglobulin VH domain comprising the amino acid sequence set forth as SEQ ID NO:8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO:5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO:5 and SEQ ID NO:6. Alternatively, disclosed herein is an IL-17 antibody (e.g. secukinumab) or an antigen-binding fragment thereof, for use in the manufacture of a medicament for treating HS, comprising subcutaneously (SC) administering to a patient in need thereof a dose of about 300 mg-about 450 mg of the IL-17 antibody or an antigen-binding fragment thereof, weekly during weeks 0, 1, 2, and 3, and thereafter SC at a dose of about 300 mg-about 450 mg: a) monthly (every 4 weeks), beginning during week 4; or b) every other week (every 2 weeks), beginning during week 4, wherein the IL-17 antibody or an antigen-binding fragment thereof comprises: i) an immunoglobulin VH domain comprising the amino acid sequence set forth as SEQ ID NO:8 and an immunoglobulin VL domain comprising the amino acid sequence set forth as SEQ ID NO:10; ii) an immunoglobulin VH domain comprising the hypervariable regions set forth as SEQ ID NO:1, SEQ ID NO:2, and SEQ ID NO:3 and an immunoglobulin VL domain comprising the hypervariable regions set forth as SEQ ID NO:4, SEQ ID NO:5 and SEQ ID NO:6; or iii) an immunoglobulin VH domain comprising the hypervariable regions set forth as SEQ ID NO:11, SEQ ID NO:12 and SEQ ID NO:13 and an immunoglobulin VL domain comprising the hypervariable regions set forth as SEQ ID NO:4, SEQ ID NO:5 and SEQ ID NO:6.

In preferred embodiments of the disclosed methods, uses and kits, the dose of the IL-17 antibody or antigen-binding fragment is about 300 mg or about 450 mg.

In preferred embodiments of the disclosed methods, uses and kits, the IL-17 antibody or antigen-binding fragment thereof is administered SC at a dose of about 300 mg weekly during weeks 0, 1, 2, and 3, and thereafter SC at a dose of about 300 mg every other week (every two weeks), beginning during week 4.

In other preferred embodiments of the disclosed methods, uses and kits, the IL-17 antibody or antigen-binding fragment thereof is administered SC at a dose of about 300 mg weekly during weeks 0, 1, 2, and 3, and thereafter SC at a dose of about 300 mg every month (every four weeks), beginning during week 4.

In preferred embodiments of the disclosed methods, uses and kits, the patient achieves a sustained response after one year of treatment, as measured by (simplified) Hidradenitis Suppurativa Clinical Response (HiSCR), Numerical Rating Scale (NRS), modified Sartorius HS score, Hidradenitis Suppurativa-Physician Global Assessment (HS-PGA), or Dermatology Life Quality Index (DLQI).

In preferred embodiments of the disclosed methods, uses and kits, prior to treatment with the IL-17 antibody or antigen-binding fragment, the patient has been previously treated with a systemic agent for HS. In preferred embodiments of the disclosed methods, uses and kits, the systemic agent is selected from the group consisting of a topical treatment, an antibiotic, an immune system suppressant, a TNF-alpha inhibitor, an IL-1 antagonist, and combinations thereof.

In some embodiments of the disclosed methods, uses and kits, prior to treatment with the IL-17 antibody or antigen-binding fragment, the patient has not been previously treated with a systemic agent or a topical treatment for HS.

In preferred embodiments of the disclosed methods, uses and kits, the IL-17 antibody or antigen-binding fragment is administered in combination with at least one of a TNF-alpha inhibitor, an antibiotic, an IL-1 inhibitor, or an immunosuppressant.

In preferred embodiments of the disclosed methods, uses and kits, the dose of the IL-17 antibody or antigen-binding fragment is about 300 mg. In other preferred embodiments of the disclosed methods, uses and kits, the dose of the IL-17 antibody or antigen-binding fragment is about 450 mg.

In preferred embodiments of the disclosed methods, uses and kits, the patient has moderate to severe HS.

In preferred embodiments of the disclosed methods, uses and kits, the patient is an adult. In some embodiments of the disclosed methods, uses and kits, the patient is an adolescent.

In preferred embodiments of the disclosed methods, uses and kits, the IL-17 antibody or antigen-binding fragment is disposed in a pharmaceutical formulation, wherein said pharmaceutical formulation further comprises a buffer and a stabilizer. In some embodiments of the disclosed methods, uses and kits, the pharmaceutical formulation is in liquid form. In some embodiments of the disclosed methods, uses and kits, the pharmaceutical formulation is in lyophilized form. In some embodiments of the disclosed methods, uses and kits, pharmaceutical formulation is disposed within pre-filled syringes, vials, injection pens, or autoinjectors.

In preferred embodiments of the disclosed methods, uses and kits, the dose of the IL-17 antibody or antigen-binding fragment is about 300 mg, the pharmaceutical formulation is disposed within means for administering selected from the group consisting of a pre-filled syringe, an injection pen, and an autoinjector, and said means is disposed within a kit, and the kit further comprises instructions for use.

In preferred embodiments of the disclosed methods, uses and kits, the dose of the IL-17 antibody or antigen-binding fragment is about 300 mg, the pharmaceutical formulation is disposed within an autoinjector or a pre-filled syringe, and the autoinjector or pre-filled syringe is disposed within a kit, and the kit further comprises instructions for use.

In preferred embodiments of the disclosed methods, uses and kits, the dose of the IL-17 antibody or antigen-binding fragment is about 300 mg, the pharmaceutical formulation is disposed within autoinjectors or pre-filled syringes, the autoinjectors or pre-filled syringes are disposed within a kit, and the kit further comprises instructions for use.

In preferred embodiments of the disclosed methods, uses and kits, the dose of the IL-17 antibody or antigen-binding fragment is about 450 mg, the pharmaceutical formulation is disposed within autoinjectors or pre-filled syringes, the autoinjectors or pre-filled syringes are disposed within a kit, and the kit further comprises instructions for use.

In preferred embodiments of the disclosed methods, uses and kits, the dose is 300 mg, which is administered as a single subcutaneous administration in a total volume of 2 ml from a formulation comprising 150 mg/ml of the IL-17 antibody or antigen-binding fragment, wherein the pharmacological exposure of the patient to the IL-17 antibody or antigen-binding fragment is equivalent to the pharmacological exposure of the patient to the IL-17 antibody or antigen-binding fragment using two separate subcutaneous administrations of a total volume of 1 ml each of the same formulation.

In preferred embodiments of the disclosed methods, uses and kits, the dose is 300 mg, which is administered as two separate subcutaneous administrations in a volume of 1 ml each from a formulation comprising 150 mg/ml of the IL-17 antibody or antigen-binding fragment

In preferred embodiments of the disclosed methods, uses and kits, prior to treatment with the IL-17 antibody or antigen-binding fragment, the patient has an HS-PGA score of ≥3. In some embodiments, the patient is selected for treatment based on having an HS-PGA score of ≥3

In preferred embodiments of the disclosed methods, uses and kits, prior to treatment with the IL-17 antibody or antigen-binding fragment, the patient is classified under Hurley stage II or III. In some embodiments, the patient is selected for treatment based on being classified under Hurley stage II or III.

In preferred embodiments of the disclosed methods, uses and kits, the patient achieves a (simplified) HiSCR by week 16 of treatment.

In preferred embodiments of the disclosed methods, uses and kits, the patient achieves an NRS30 by week 16 of treatment.

In preferred embodiments of the disclosed methods, uses and kits, the patient has a reduction in HS flares by week 16 of treatment.

In preferred embodiments of the disclosed methods, uses and kits, the patient achieves a reduction of ≤6 as measured by the DLQI by week 16 of treatment.

In preferred embodiments, when the disclosed methods, uses or kits are employed to treat a population of patients with moderate to severe HS, at least 51% of said patients achieve a simplified HiSCR by week 16 of treatment in response to said administering step.

In preferred embodiments, when the disclosed methods, uses or kits are employed to treat a population of patients with moderate to severe HS, at least 40% of said patients achieve an NRS30 response by week 16 of treatment in response to said administering step.

In preferred embodiments, when the disclosed methods, uses or kits are employed to treat a population of patients with moderate to severe HS, less than 15% of said patients experience an HS flare during 16 weeks of treatment in response to said administering step.

In preferred embodiments of the disclosed methods, uses and kits, prior to treatment with the IL-17 antibody or antigen-binding fragment thereof, the patient does not have extensive scarring (<20 fistulas) as a result of HS. In some embodiments, the patient is selected for treatment based on not having extensive scarring (<20 fistulas) as a result of HS.

In preferred embodiments of the disclosed methods, uses and kits, the patient is additionally treated with at least one topical medication and at least one antiseptic in combination with the IL-17 antibody or antigen-binding fragment thereof.

In preferred embodiments of the disclosed methods, uses and kits, the patient is treated with the IL-17 antibody or antigen-binding fragment thereof for at least one year.

In preferred embodiments of the disclosed methods, uses and kits, the patient has a rapid reduction in pain, as measured by VAS or NRS, as early as one week after the first dose of the IL-17 antibody or antigen-binding fragment thereof.

In preferred embodiments of the disclosed methods, uses and kits, the patient has a rapid reduction in CRP, as measured using a standard CRP assay, as early as one week after the first dose of the IL-17 antibody or antigen-binding fragment thereof.

In preferred embodiments of the disclosed methods, uses and kits, the patient has a reduction in modified Sartorius score by 16 weeks of treatment.

In preferred embodiments of the disclosed methods, uses and kits, the patient has an improvement in DLQI by 16 weeks of treatment.

In preferred embodiments of the disclosure, the IL-17 antibody or antigen-binding fragment thereof is a monoclonal antibody.

In preferred embodiments of the disclosure, the IL-17 antibody or antigen-binding fragment thereof is a human or humanized antibody.

In preferred embodiments of the disclosure, the IL-17 antibody or antigen-binding fragment thereof is a human antibody.

In preferred embodiments of the disclosed methods, uses and kits, the IL-17 antibody or antigen-binding fragment is a human monoclonal antibody.

In preferred embodiments of the disclosure, the IL-17 antibody or antigen-binding fragment thereof is a human antibody of the IgG₁ subtype.

In preferred embodiments the IL-17 antibody or antigen-binding fragment thereof has a kappa light chain.

In preferred embodiments of the disclosure, the IL-17 antibody or antigen-binding fragment thereof is a human antibody of the IgG₁ kappa type.

In preferred embodiments of the disclosed methods, uses and kits, the IL-17 antibody or antigen-binding fragment has a Tₘₐₓ of about 7-8 days.

In preferred embodiments of the disclosed methods, uses and kits, the IL-17 antibody or antigen-binding fragment has an absolute bioavailablilty of about 60%-about 80%.

In preferred embodiments of the disclosure, the IL-17 antibody or antigen-binding fragment thereof is secukinumab.

### Kits

The disclosure also encompasses kits for treating HS. Such kits comprise an IL-17 antagonist, e.g., IL-17 binding molecule (e.g., IL-17 antibody or antigen-binding fragment thereof, e.g., secukinumab) or IL-17 receptor binding molecule (e.g., IL-17 antibody or antigen-binding fragment thereof) (e.g., in liquid or lyophilized form) or a pharmaceutical composition comprising the IL-17 antagonist (described supra). Additionally, such kits may comprise means for administering the IL-17 antagonist (e.g., an autoinjector, a syringe and vial, a prefilled syringe, a prefilled pen) and instructions for use. These kits may contain additional therapeutic HS agents (described supra) for treating HS, e.g., for delivery in combination with the enclosed IL-17 antagonist, e.g., IL-17 binding molecule, e.g., IL-17 antibody, e.g., secukinumab. Such kits may also comprise instructions for administration of the IL-17 antagonist (e.g., IL-17 antibody, e.g., secukinumab) to treat the HS patient. Such instructions may provide the dose (e.g., 10 mg/kg, 300 mg, 450 mg), route of administration (e.g., IV, SC), and dosing regimen (e.g., weekly, monthly, weekly and then monthly, weekly and then every other week, etc.) for use with the enclosed IL-17 antagonist, e.g., IL-17 binding molecule, e.g., IL-17 antibody, e.g., secukinumab.

The phrase “means for administering” is used to indicate any available implement for systemically administering a drug to a patient, including, but not limited to, a pre-filled syringe, a vial and syringe, an injection pen, an autoinjector, an IV drip and bag, a pump, etc. With such items, a patient may self-administer the drug (i.e., administer the drug without the assistance of a physican) or a medical practitioner may administer the drug. In some embodiments, a total dose of 300 mg is to be delivered in a total volume of 2 ml, which is disposed in two PFSs or autoinjectors, each PFS or autoinjector containing a volume of 1 ml having 150 mg/ml of the IL-17 antibody, e.g., secukinumab. In this case, the patient receives two 1 ml injections (a multi-dose preparation). In preferred embodiments, a total dose of 300 mg is to be delivered in a total volume of 2 ml having 150 mg/ml of the IL-17 antibody, e.g., secukinumab, which is disposed in a single PFS or autoinjector. In this case, the patient receives one 2 ml injection (a single dose preparation).

Disclosed herein are kits for use treating a patient having HS, comprising an IL-17 antagonist (e.g., IL-17 binding molecule, e.g., IL-17 antibody or antigen-binding fragment thereof, e.g., secukinumab) and means for administering the IL-17 antagonist to the HS patient. In some embodiments, the kit further comprises instructions for administration of the IL-17 antagonist, wherein the instructions indicate that the IL-17 antagonist (e.g., IL-17 binding molecule, e.g., IL-17 antibody or antigen-binding fragment thereof, e.g., secukinumab) is to be administered to the patient:
1) SC at about 300 mg-about 450 mg (e.g., about 300 mg, or about 450 mg) weekly during weeks 0, 1, 2, and 3, and thereafter SC at about 300 mg-about 450 mg (e.g., about 300 mg, about 450 mg):
   a) monthly (every 4 weeks), beginning during week 4; or
   b) every other week (every 2 weeks), beginning during week 4;
2) IV at about 3 mg/kg-about 10 mg/kg (e.g., about 3 mg/kg, about 10 mg/kg) every other week during weeks 0, 2, and 4, and thereafter SC at about 300 mg-about 450 mg (e.g., about 300 mg, about 450 mg):
   a) monthly (every 4 weeks), beginning during week 8; or
   b) every other week (every 2 weeks), beginning during week 6.

### General

In the most preferred embodiments of the disclosed uses, methods and kits, the IL-17 antagonist is an IL-17 antibody or antigen-binding fragment thereof. In some embodiments of the disclosed uses, methods and kits, the IL-17 antibody or antigen-binding fragment thereof is selected from the group consisting of: a) an IL-17 antibody or antigen-binding fragment thereof that binds to an epitope of human IL-17 comprising Leu74, Tyr85, His86, Met87, Asn88, Va1124, Thr125, Pro126, Ile127, Va1128, His129; b) an IL-17 antibody or antigen-binding fragment thereof that binds to an epitope of human IL-17 comprising Tyr43, Tyr44, Arg46, Ala79, Asp80; c) an IL-17 antibody or antigen-binding fragment thereof that binds to an epitope of an IL-17 homodimer having two mature human IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain; d) an IL-17 antibody or antigen-binding fragment thereof that binds to an epitope of an IL-17 homodimer having two mature human IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody or antigen-binding fragment thereof has a K_{D} of about 100-200 pM, and wherein the IL-17 antibody or antigen-binding fragment thereof has an in vivo half-life of about 23 to about 35 days; e) an IL-17 antibody that binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system (e.g., Biacore), and wherein the IL-17 antibody has an in vivo half-life of about 23 to about 30 days; and f) an IL-17 antibody or antigen-binding fragment thereof comprising: i) an immunoglobulin heavy chain variable domain (V_{H}) comprising the amino acid sequence set forth as SEQ ID NO: 8; ii) an immunoglobulin light chain variable domain (V_{L}) comprising the amino acid sequence set forth as SEQ ID NO:10; iii) an immunoglobulin V_{H} domain comprising the amino acid sequence set forth as SEQ ID NO:8 and an immunoglobulin V_{L} domain comprising the amino acid sequence set forth as SEQ ID NO:10; iv) an immunoglobulin V_{H} domain comprising the hypervariable regions set forth as SEQ ID NO:1, SEQ ID NO:2, and SEQ ID NO:3; v) an immunoglobulin V_{L} domain comprising the hypervariable regions set forth as SEQ ID NO:4, SEQ ID NO:5 and SEQ ID NO:6; vi) an immunoglobulin V_{H} domain comprising the hypervariable regions set forth as SEQ ID NO:11, SEQ ID NO:12 and SEQ ID NO:13; vii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; viii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6; ix) an immunoglobulin light chain comprising the amino acid sequence set forth as SEQ ID NO:14; x) an immunoglobulin heavy chain comprising the amino acid sequence set forth as SEQ ID NO:15; or xi) an immunoglobulin light chain comprising the amino acid sequence set forth as SEQ ID NO:14 and an immunoglobulin heavy chain comprising the amino acid sequence set forth as SEQ ID NO:15.

In most preferred embodiments of the disclosed methods, kits, or uses, the IL-17 antibody or antigen-binding fragment thereof is a monoclonal antibody. In most preferred embodiments of the disclosed methods, kits, or uses the IL-17 antibody or antigen-binding fragment thereof is a human or humanized antibody, preferably a human antibody. In most preferred embodiments of the disclosed methods, kits, or uses, the IL-17 antibody or antigen-binding fragment thereof is a human antibody of the IgG₁ isotype. In most preferred embodiments of the disclosed methods, kits, or uses, the antibody or antigen-binding fragment thereof is secukinumab.

In most preferred embodiments of the disclosed methods, kits, or uses, the antibody or antigen-binding fragment thereof is secukinumab, the dose size is flat (also referred to as a “fixed” dose, which differs from weight-based or body surface area-based dosing), the dose is 300 mg, the route of administration is SC, and the regimen is administration at week 0, 1, 2, 3, 4, 6, 8, 10, 12 etc. (weekly during week 0, 1, 2, and 3, and then every other week, beginning during week 6).

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

### Example 1: Early Studies of Anti-IL-17 Antibodies in Treating HS

Early clinical evidence of the effects of an anti-IL-17 antibody, CJM112, supports the potential of an anti-IL-17 antibody as an effective therapy for patients with HS. Like secukinumab, CJM112 is a recombinant fully human anti-interleukin-17A monoclonal antibody of the IgG1/κ isotype, developed for the potential treatment of autoimmune and inflammatory conditions. CJM112 binds with higher affinity to human homodimer IL-17A (6 pM) than secukinumab, and neutralizes the bioactivity of IL-17A in vitro.

This phase 2 study (CCJM112X2202) was a randomized, placebo controlled, double blind, multicenter study with two periods in patients with moderate to severe chronic HS in parallel groups conducted in USA, Denmark, Switzerland, Germany and the Netherlands. This study consisted of approximately 4 weeks screening period, two sequential treatment periods of 16 weeks (Period 1 and Extension Period 2) and approximately 12 weeks of follow-up with no treatment. Patients were randomized in a 2:1:1 ratio to one of the following three treatment sequences:
Treatment sequence 1: CJM112 300 mg s.c. in Period 1; placebo s.c. in Extension Period 2
Treatment sequence 2: Placebo s.c. in Period 1; CJM112 50 mg s.c. in Extension Period 2
Treatment sequence 3: Placebo s.c. in Period 1; CJM112 300 mg s.c. in Extension Period 2

During each treatment period, patients were dosed a total of 10 times. Study medication was administered subcutaneously (s.c.) at the clinical center. The investigational drug, CJM112 150 mg/ml and matching placebo was provided in glass vials each containing 150 mg CJM112 or placebo as a liquid. In each treatment period, the first five doses were administrated weekly, followed by five bi-weekly administrations.

The chosen primary endpoint was the clinical response rate (based on responders defined as a 2 point reduction in Hidradenitis Suppurativa-Physician Global Assessment (HS PGA) score from baseline). The HS-PGA score is a static global severity 6-point scale which is described in Kimball et al 2012 Ann Intern Med 157:846-855.

The study population comprised of adult male and female patients with clinically diagnosed chronic HS for at least 1 year (prior to screening), who had undergone previous antibiotic therapy, had a HS-PGA score of at least moderate severity (score of 3 or higher of a 6-point scale) at baseline, at least 4 inflammatory abscesses and/or inflammatory nodules (AN) present in at least two distinct anatomical areas, and at least one area had to be minimally Hurley Stage II (moderate) at baseline. No more than 25 draining fistulae were accepted for eligibility at baseline.

In addition, body weight needed to be between 50 and 150 kg.

Exclusion criteria included previous treatment with biologics agents that block IL-17 or IL-17R, including secukinumab, ixekizumab and brodalumab, recent use of other biologics (e.g. adalimumab within the last 3 months), use of any systemic treatment for HS in the last 4 weeks prior to randomization (such as retinoids or other immunomodulating), or use of systemic antibiotics for HS in the last week prior to randomization/first treatment. If spironolactone or other antiandrogenes (such as finasteride, cyproterone acetate, etc.) were used (for HS), only patients with a stable dose in the last 3 months and planning to continue for the duration of the study are eligible. Patients with a history of severe systemic Candida infections or evidence of Candidiasis in the last two weeks prior to inclusion as well as patients with active systemic or skin infections (other than common cold or HS-related) during the two weeks before randomization/first treatment needed to be excluded. Additional exclusion criteria did apply. Topical antibiotics and antiseptics, as well as standard wound care were allowed throughout the study. Oral antibiotics could be used as rescue treatment in case of skin infection, but should not have been used longer than 2 weeks.

A total of 66 patients were enrolled and randomized into Period 1 (33 in CJM112 300 mg group and 33 in placebo group), of which 60 (90.9%) patients (29 in CJM112 300 mg group and 31 in placebo group) completed Week 16 in Period 1. A total of six patients discontinued from the study before Week 16. The reasons for study discontinuations were “lost to follow-up”, “adverse event” (cystitis in the CJM112 group) and “patient/guardian decision”.

The primary endpoint was to determine the efficacy of CJM112 300 mg in HS patients by using the clinical responder rate at Week 16 (end of Period 1), in comparison to placebo. The HS-PGA (Hidradenitis Suppurativa-Physician Global Assessment, see Kimball 2012) responder rate, defined as an at least 2 point reduction from baseline on a 6-point scale, was used for that purpose.

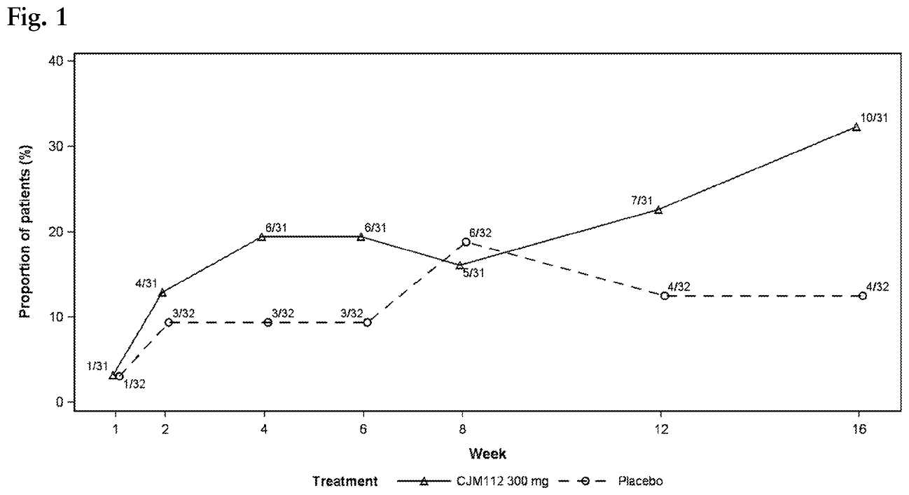

The HS-PGA responder rate is shown in FIG. 1 and Bayesian statistics was used to compare treatments at Week 16 (Table 4).

TABLE 4
Treatment comparison (Bayesian model) of HS-PGA responder
rate at Week 16-Period 1 (PD analysis set 1)

| | | Posterior difference | Posterior probability of |
|---|---|---|---|
| | Posterior response | CJM112 300 mg- | CJM112 300 mg |
| | proportion (95% | placebo (95% | superiority to placebo |
| Treatment | Credibility Interval) | Credibility Interval) | group |
| CJM112 300 mg | 0.326 [0.177; 0.496] | 0.194 [−0.005; 0.390] | 0.9723 |
| Placebo | 0.133 [0.041; 0.267] | | |

The proportion of patients reaching this endpoint at Week 16 (HS-PGA responder rate) was 32.3% responders (or 10 out of 31) with anti-IL17 treatment versus 12.5% (or 4 out of 32) with placebo. Posterior probability in favor of CJM112 was as high as 0.97 indicating that this may reflect a true effect.

The primary endpoint of this study was slightly different to the primary endpoint of the previously conducted phase 2 study using HUMIRA (Kimball et al 2012 Ann Intern Med 157:846-855). While the same 6-point HS-PGA score was used, the definition of responders varied across studies. Kimball used a more stringent and composite definition, where a 2-point reduction from baseline AND clear/minimal/mild stage at week 16 was needed to be achieve responder status.

For comparison purposes we calculated the responder rate score with the “Kimball 2012” definition. Six out of 31 patients responded (19.4%) in the CJM112 group and only 2 out of 32 (6.3%) in the placebo treated group. This is comparable to what adalimumab achieved in the phase 2 study (Table 5).

TABLE 5
Comparison between phase 2 studies in moderate to severe HS using
therapeutic antibodies: Adalimumab (HUMIRA) and CJM112

| | | Adalimumab | | | |
|---|---|---|---|---|---|
| | | (HUMIRA) | | CJM112 Ph2a | |
| | | Ph2b (Kimball 2012) | | X2202 | |
| | | N = 154 (3 arms) | | N = 66 (2 arms) | |
| | Definition | Ada (high) | Placebo | CJM112 | Placebo |
| HS PGA | 2 pt reduction | 17.6% | 3.9% | 19.4% | 6.3% (2/32) |
| responder rate | AND | (9/51) | (2/51) | (6/31) | |
| (week 16) | clear/minimal/mild | | | | |
| HiSCR responder* | | 61% | 16% | 39% | 38% |
| (wk 16) | | | | | |
| Inflammatory | Infl. Nodules | −43% | −13% | −55.0% | −27.6% |
| lesions percent | Abscesses | −58% | −27% | −33.4% | −63.3% |
| reduction from | Fistulae | −31% | +25% | −59.0% | +62.5% |
| baseline (mean, | | | | | |
| week 16) | | | | | |
| QoL (week 16) | DLQI (change | −6.0 | −1.9 | −5.9 | −2.8 |
| | from baseline) | | | | |

*post-hoc analysis for HUMIRA phase2b at week 12 (HUMIRA EPAR)

In addition to the primary endpoint (HS-PGA), numerical decreases in inflammatory lesions, mainly in inflammatory nodules, were observed in both treatment arms, CJM112 and placebo, with larger magnitude decreases in the CJM112 group. The magnitude of the treatment effect was similar to that observed with adalimumab (−2.6 inflammatory lesions in CJM112 relative to placebo vs-2.4 to-3.0 in adalimumab relative to placebo). However, the treatment effect in reducing inflammatory lesions was not statistically significant in this small phase 2 study.

In order to better understand the clinical relevance of the effects on inflammatory lesion counts observed in the CJM112 phase 2 study, the data has been compared to the publicly available data from adalimumab (Table 6). Absolute lesion count results are only publicly available from the phase 3 studies. PIONEER 1 recruited a more severe population, closer to the CJM112 phase 2 study, than PIONEER 2 and therefore PIONEER 1 is used for the comparison below.

TABLE 6
Change from Baseline to Week 12 in number of inflammatory
lesions by lesion type compared to HUMIRA phase 3 PIONEER
1 study (without antibiotics)

| | | HUMIRA Ph3 | | CJM112 Ph2a | |
|---|---|---|---|---|---|
| | | [Study PIONEER 1] | | X2202, Week 12 | |
| | | Adalimumab | Placebo | CJM112 | Placebo |
| Lesion type | | N = 153 | N = 151 | N = 33 | N = 33 |
| Inflammatory | Mean | −4.2 | −1.9 | −7.2 | −2.3 |
| nodules | | | | | |
| Abscesses | Mean | −1.2 | −0.8 | −1.1 | −0.4 |
| Draining fistulae | Mean | −0.8 | −0.3 | −0.7 | −0.1 |

Although population of the CJM112 phase 2 study had a higher number of inflammatory lesions at baseline than the PIONEER 1 study, the magnitude of changes observed in inflammatory lesion counts during the study were similar between the placebo groups of both studies, supporting that the population entering the CJM112 phase 2 was representative of a moderate to severe HS population who behaved within the expected variability during the study.

Treatment effects for adalimumab and CJM112 were mainly observed in the inflammatory lesion counts and were of slightly larger magnitude with CJM112 than with adalimumab. Only small treatment effects were observed in abscesses and draining fistulae in both studies. Consistent results were also observed in PIONEER 2 (Kimball et al 2016a N Engl J Med; 375(5):422-434.).

No significant clinical safety signal was detected, despite use of a relatively high dose of IL-17 blockade (first five CJM112 300 mg doses were administrated weekly, followed by five bi-weekly administrations). The majority of the AEs were mild or moderate in severity. Overall infections were at the same frequency in the active groups than in placebo treated group, though some infections were encountered more often in the CJM112 treated patients (such as nasopharyngitis, upper respiratory tract infection, cystitis). Whereas, adverse events had differences in frequencies between groups, the low numbers of patients do not permit to conclude in this rather small population. Serious adverse events were only observed in placebo treated periods and considered not related (angina pectoris and abscess). The latter was encountered approx. Twenty-three weeks after the last dose of CJM112 and was a hospitalization for a removal of an HS abscess.

No new or unexpected safety signal could be detected in the HS population after treatment with a high affinity anti-IL-17A antibody, CJM112.

### Example 2

### Example 2A: Responder Rate Predictions in Heavy Subjects for Higher Dosage (450 mg) and More Frequent Dosing (Q2w) of Secukinumab

The purpose of the modeling and simulation (M&S) work in this Example is to investigate the simulated efficacy of secukinumab in heavy subjects following the two higher dosage regimens mentioned above, 450 Q4W and 300 Q2W. We report here modeling and simulation (M&S) work that investigated responder rate of PASI 75 and PASI 90 in patients ≥90 kg in bodyweight, using the standard regimen for secukinumab in psoriasis, i.e. 300 mg Q4W, in comparison to predicted response using higher dose regimens, i.e. 450 mg Q4W or 300 mg Q2W. The main objective of the work is to use model predicted (i.e. simulated) response rates to estimate the magnitude of improvement with the higher doses in heavier patients.

PASI data from studies CAIN457A2302 and CAIN457A2303 up to week 52 were used in this analysis. Only subjects ≥90 kg were used in the model building (n=641). The data included responses from both 150 mg and 300 mg regimens, in addition to patients who were initially treated with placebo up to week 12 and subsequently randomized to 150 mg or 300 mg. The response variable, either PASI 75 or PASI 90, a binary outcome, was modeled as a function of serum secukinumab concentration. Due to the lag time between response and concentration, an indirect response model was used. All measurements up to week 52 were used in the model. Predicted concentration at times of PASI measurements were used, calculated from post-hoc estimates of a previously developed secukinumab population pharmacokinetic (PK) model.

In brief, the model was a two compartment model with first order absorption, and bodyweight as a covariate on the central clearance (CL), central and peripheral compartment volumes (V2 and V3), and inter-compartmental clearance (Q). Post hoc estimates for the PK model parameters for the patients in A2302 and A2303 were used as input into the Pharmacodynamic (PD) modeling. Previous modeling efforts for secukinumab in psoriasis also included a population PK/PD model for continuous PASI score. However, this model had some limitations to describe the PASI 75 responder rates (e.g. slight over—prediction during the induction phase), and this was more pronounced with more extreme response thresholds like PASI 90. Covariate search (such as baseline PASI or body weight) was also previously investigated and was found not to improve model fit, therefore no covariate search was implemented here.

Two components of a population model are: the structural model, which accounts for the systematic trends in the data and, to the extent possible, the mechanisms generating those trends; and the random effects model, which accounts for inter and intra-subject variability about those trends. In this analysis, the model components were selected and assembled based on a combination of prior knowledge, modeling experience with PASI response of secukinumab, and data driven decision-making guided by statistical and heuristic rules. The analysis was performed using the NONMEM software system, NONMEM version 7.3.0 (Icon Development Solutions, Ellicott City, Md., USA), utilizing the MODESIM high performance computing environment accessed from GPSII. Perl-speaks-NONMEM 4.2.0 was used for run automation. All model building was performed using the Laplace method. The analysis was performed to estimate the population parameters (mean and between subject variability).

After inspection of model diagnostics, a final (best) model was selected on the basis of likelihood and Bayesian Information Criteria (BIC). Using the best model, simulation was used to predict PASI 75 or PASI 90 responder rates. For each regimen (i.e. 300 mg Q4W, 300 mg Q2W, and 450 mg Q4W), 1000 replicates were generated using NONMEM's ONLYSIMULATION option. The simulations were generated using the final estimates for the fixed effect parameters (i.e. emax, ec50, kout, gamma, and alpha) and sampling inter-individual variability. PASI 75 or PASI 90 binary response was simulated by sampling from a binary distribution with probability determined from the model. The source data set for the simulations was the same data set used in establishing the model, i.e. using subjects with bodyweight ≥90 kg, and including the subjects' post-hoc PK estimates. Regular dosing schedules for 300 mg Q4W, 300 mg Q2W and 450 mg Q4W (once a week up to week 4 and then every four weeks for Q4W or every two weeks for Q2W up to week 52) were included in the simulation dataset along with a regular sampling schedule, i.e. once a week up to week 12 and every four weeks thereafter. The predicted responder rate was calculated for each simulation replicate and at each time point. From the 1000 runs, the median and 95% prediction intervals for responder rates were determined.

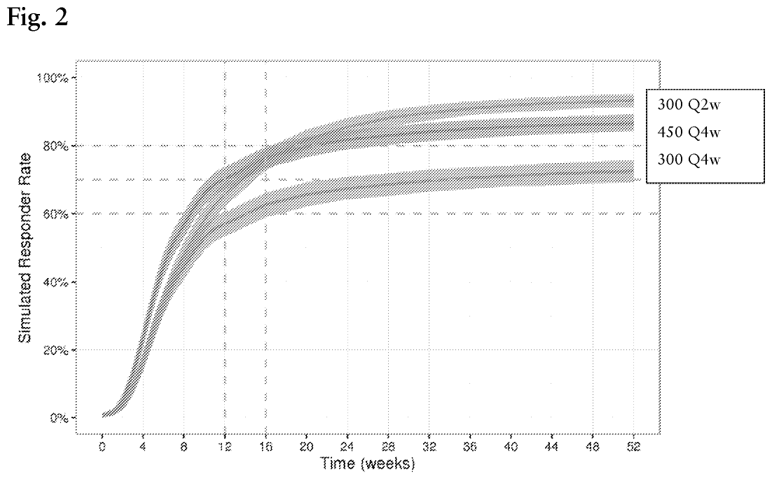

FIG. 2 shows simulated PASI 90 responder rates for different regimens in subjects with bodyweight greater or equal 90 kg. Table 7 contains the predicted responder rates for PASI 75 and PASI 90 at weeks 12, 16 and 52 for the different regimens.

TABLE 7
shows PASI 75 and PAST 90 predicted responder
rates (%) for different regimens in subjects with bodyweight
greater or equal 90 kg. Displayed as median (95% PI).

| Weight (kg) | PASI | Time (Week) | 300 Q4W | 450 Q4W | 300 Q2W |
|---|---|---|---|---|---|
| ≥90 | 75 | 12 | 80 (77, 83) | 87 (84, 89) | 85 (82, 87) |
| ≥90 | 75 | 16 | 84 (81, 87) | 91(88, 93) | 91(88, 93) |
| ≥90 | 75 | 52 | 90 (88, 92) | 96 (95, 98) | 99 (98, 99) |
| ≥90 | 90 | 12 | 57 (53, 61) | 70 (67, 74) | 66 (62, 69) |
| ≥90 | 90 | 16 | 63 (59, 66) | 76 (73, 80) | 76 (73, 79) |
| ≥90 | 90 | 52 | 73 (69, 76) | 87 (84, 89) | 93 (91, 95) |

Simulations predict that a higher responder rate (for PASI 75 or PASI 90) in heavy subjects is likely following either of the higher dosage regimens, 300 mg Q2W or 450 mg Q4W, as compared to the standard 300 mg Q4W regimen. The simulations suggest that responder rates at week 16 will be similar for either of the higher dosing regimens (91% for PASI 75 and 76% for PASI 90, see Table 7). However, compared to 450 mg Q4W, the 300 mg Q2W regimen is predicted to give higher response rates starting from week 20. By week 52 the median responder rates following 300 mg Q2W vs 450 mg Q4W are predicted to be 99% vs 96% for PASI 75 and 93% vs 87% for PASI 90 (see Table 7).

Based on this information, it is expected that HS patients, who tend to be heavier patients, and who have deep tissue disease, may benefit from more frequent administration of secukinumab or higher doses of secukinumab.

### Example 2B: Secukinumab Dose-Response Modelling and Simulation for Heavy Patients

The modeling and simulation in this example consists of week 52 data from the secukinumab OPTIMIZE study. OPTIMIZE (NCT02409667) was a 52 week comparative, randomized, multicenter, open-label trial with blinded-assessment to evaluate the efficacy, safety and tolerability of secukinumab 300 mg SC in long-term treatment optimization in patients with moderate to severe chronic plaque-type psoriasis. In this study, suboptimal responders at Week 24, i.e., patients who reached PASI75 (i.e., a 75% reduction from baseline in PASI score) but did not reach PASI90 after 24 weeks under secukinumab 300 mg q4w were subsequently randomized to either secukinumab 300 mg q4w or secukinumab 300 mg q2w until Week 52.

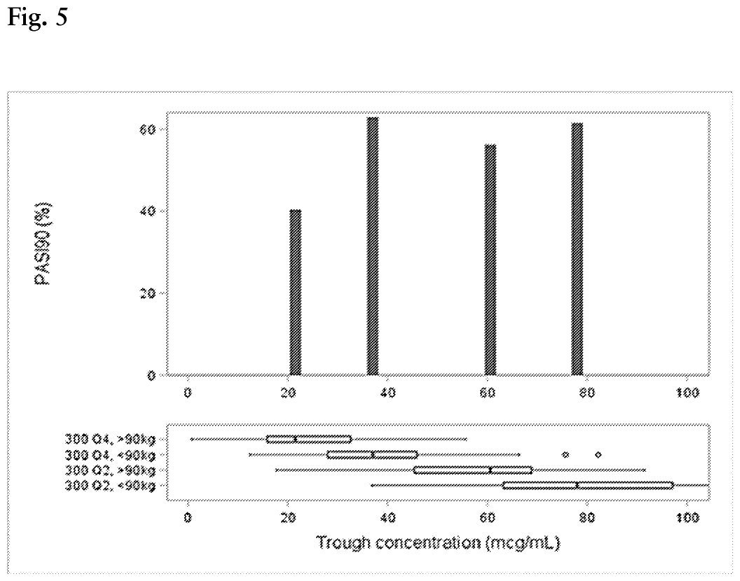

The top panel of FIG. 5 displays the percentage of responders (Patients achieving PASI90, i.e., a 90% reduction from baseline in PASI score at Week 52) by treatment group (q2w or q4w) and weight category (<90 or >90 kg) in that partial subgroup. The lower panel represents the secukinumab trough concentration (given as mcg/mL) at Week 52 in the same subgroup.

Simulation shows that heavier patients (>90 kg) benefit from being dosed 300 mg Q2W, not only in terms of exposure, but also in efficacy. PASI % response shows that the higher antibody concentration improves efficacy in patients that are >90 kg. However, the same is not seen in patients that are less than 90 kg. The data suggests that above ˜30 mcg/mL, efficacy in psoriasis patients is maximized (PASI 90˜60%). Based on this information, it is expected that HS patients, who tend to be heavier patients, and who have deep tissue disease, may benefit from more frequent administration of secukinumab.

### Example 3: Efficacy and Safety of Secukinumab in Adult Patients with Moderate to Severe HS

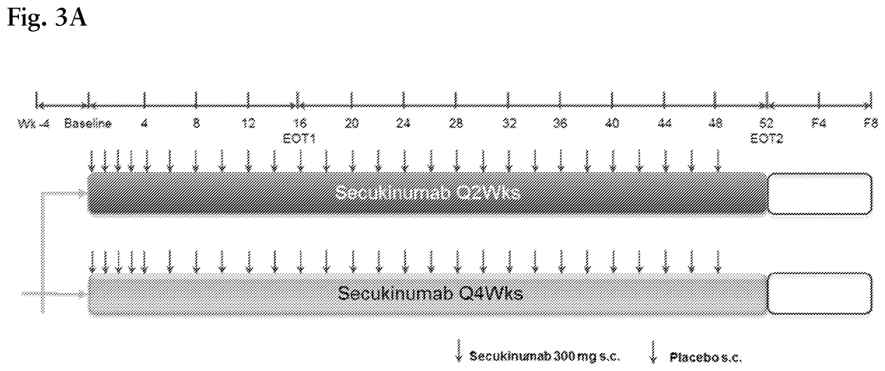

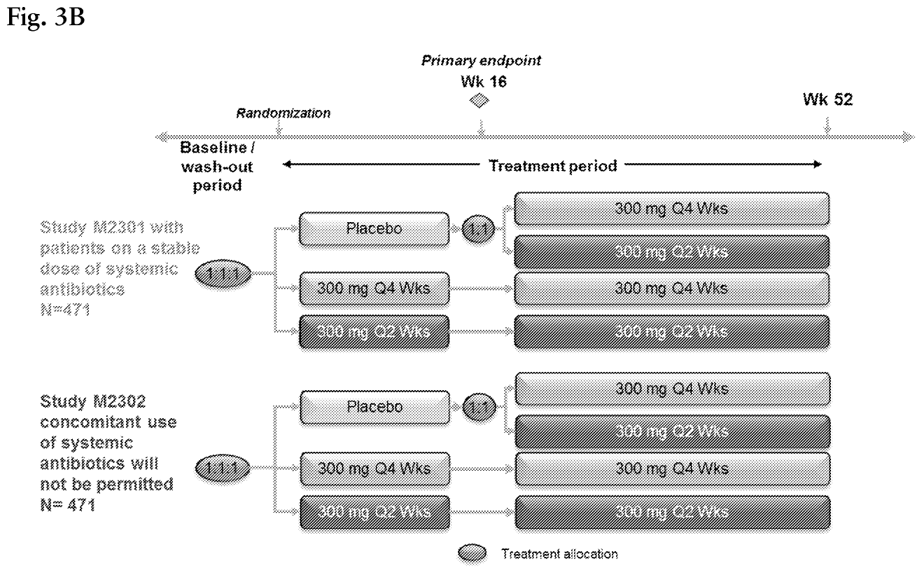

Table 8, below, sets forth details of the clinical trial design to demonstrate the efficacy of two secukinumab dose regimens compared to placebo by assessing the proportion of subjects achieving HiSCR after 16 weeks of treatment.

| | Objective | Endpoints |
|---|---|---|
| Primary | To demonstrate the efficacy of | Achievement of HiSCR at Week 16. HiSCR is at |
| Objective & | secukinumab compared to placebo | least a 50% decrease in Abscess and |
| endpoints | with respect to HiSCR after 16 weeks | Inflammatory Nodule (AN) number with no |
| | of treatment. | increase in the number of abscesses or in the |
| | | number of draining fistulas. |
| Secondary | To demonstrate the efficacy of | Flaring up to Week 16. Flare is at least a 25% |
| Objectives(s) | secukinumab compared to placebo | increase in AN counts with a minimum |
| & endpoint(s) | with respect to: | increase of 2 AN relative to baseline. |
| | proportion of patients with HS | Achievement of NRS30 at Week 16, among |
| | flares | subjects with baseline NRS ≥ 3. NRS30 is |
| | proportion of patients with | at least a 30% reduction from baseline in |
| | clinical response in HS related | Patient's Global Assessment of Skin Pain-at |
| | skin pain after 16 weeks of | worst. |
| | treatment. | |
| Exploratory | To evaluate the effect of | Absolute and percent change from baseline in |
| Objective(s) | secukinumab with respect to the | Modified Sartorius Score (mSS). |
| & endpoint(s) | following efficacy assessments: | HS-PGA response. HS-PGA response is |
| | Modified Sartorius Score; | defined as the achievement of at least a 2-point |
| | HS-Physician's Global Assessment | reduction in HS-PGA score compared to baseline. |
| | (HS-PGA); | DLQI response and absolute/percent DLQI total |
| | Dermatology Life Quality Index | score change from baseline. DLQI response is |
| | (DLQI); | defined as decrease greater than 5.0 points from |
| | Health Status Questionnaire (EQ- | baseline. |
| | 5D-3L); | EQ-5D-3L Categories on Category questions |
| | Patient Global Impression of severity | and summary statistics on EQ-5D-3L score |
| | (PGI-s); | questions. |
| | Patient Global Impression of change | Patient Global Impression of severity and |
| | (PGI-c); | change (PGI-s and PGI-c) categories. |
| | Work Productivity Activity | Absolute and percent change from baseline in |
| | Impairment (WPAI); | Work Productivity and Activity Impairment- |
| | HS Symptom Diary | Specific Health Problem (WPAI-SHP). |
| | compared to placebo after 16 weeks | HS Symptom Diary items score change from |
| | and in the two secukinumab dose | baseline. |
| | regimens up to 52 weeks of treatment | Achievement of clinical response as defined by |
| | To explore the long-term effect of | HiSCR. |
| | secukinumab with respect to HiSCR, | Flares |
| | proportion of patients with flares, HS | Achievement of pain relief as defined by NRS30 |
| | related skin pain up to 52 weeks of | Clinical safety and tolerability assessments |
| | treatment. | anti-AIN457 antibodies levels in serum |
| | To evaluate the safety and | Biomarkers in serum |
| | tolerability of secukinumab over 52 | |
| | weeks of treatment. | |
| | To evaluate the pharmacokinetics of | |
| | secukinumab in HS patients. | |
| | To assess the development of | |
| | immunogenicity against secukinumab | |
| | To explore the potential association | |
| | of biomarker levels with secukinumab | |
| | efficacy and safety by visit up to end | |
| | of study. | |

| | |
|---|---|
| Inclusion | In addition to the standard inclusion criteria, the following will be implemented: |
| Criteria | 1. Male and female patients ≥ 18 years of age. |
| | 2. Diagnosis of HS ≥ 1 year prior to baseline. |
| | 3. Patients with moderate to severe HS defined as: |
| | A total of at least 5 inflammatory lesions, i.e. abscesses and/or inflammatory |
| | nodules |
| | Inflammatory lesions should affect at least 2 distinct anatomic areas |
| | Patients must agree to daily use of topical over-the-counter antiseptics on the areas |
| | affected by HS lesions. |
| | For study M2301, subjects should be on a stable dose of permitted oral antibiotics for at |
| | least 28 days prior to randomization and stay on that stable dose for at least 16 weeks. |
| | For study M2302, oral antibiotics for treating HS are not allowed during the study. |
| | Note: Previous failure to antibiotic therapy not required to enter the studies |
| Exclusion | In addition to the standard exclusion criteria, the following will be implemented: |
| Criteria | HS specific criteria: |
| | 1. Total fistulae count ≥ 20 at baseline. |
| | 2. Any other active skin disease or condition that may interfere with assessment of HS. |
| | 3. Active ongoing inflammatory diseases other than HS that require treatment with |
| | prohibited medications. |
| | 4. Underlying conditions (including, but not limited to metabolic, hematologic, renal, |
| | hepatic, pulmonary, neurologic, endocrine, cardiac, infectious or gastrointestinal) which |
| | in the opinion of the investigator significantly immunocompromises the subject and/or |
| | places the subject at unacceptable risk for receiving an immunomodulatory therapy. |
| | 5. Current severe progressive or uncontrolled diseases which renders the patient |
| | unsuitable for the trial or puts the patient at increased risk, including any medical or |
| | psychiatric condition which, in the Investigator's opinion, would preclude the participant |
| | from adhering to the protocol or completing the study per protocol. |
| | 6. Use or planned use of prohibited treatment. Washout periods detailed in the protocol |
| | have to be adhered to. |
| | 7. For patients enrolling in the non-antibiotic strata: use of systemic antibiotics for the |
| | treatment of HS within 28 days before baseline. |
| | For patients enrolling in the antibiotic strata: patients enter the study under concomitant |
| | treatment with systemic antibiotics (as per protocol) on a stable dose (defined as a dose |
| | or dose regimen that has not changed in the previous 28 days before baseline and is |
| | considered unlikely to change at least for the first 16 weeks during the study). |
| | 8. History of hypersensitivity to any of the study drug constituents. |
| | 9. Previous exposure to secukinumab (AIN457) or any other biologic drug directly |
| | targeting IL-17 A/F or the IL-17 receptor. |
| | 10. History of chronic or recurrent systemic infections or active systemic infections during |
| | the last two weeks (exception: common cold) prior to randomization. |
| | 11. Evidence of tuberculosis infection as defined by a positive QuantiFERON ® TB-Gold |
| | test (QFT) at screening. Subjects with a positive or indeterminate QFT test may |
| | participate in the study if a full tuberculosis work-up (according to local |
| | practice/guidelines) completed within 12 weeks prior to randomization, establishes |
| | conclusively that the subject has no evidence of active or latent tuberculosis. |
| | 12. Medical history record of infection with HIV, hepatitis B or C prior to randomization, |
| | except for hepatitis C successfully treated and cured. |
| | 13. History of lymphoproliferative disease or any known malignancy or history of |
| | malignancy of any organ system treated or untreated within the past 5 years, regardless |
| | of whether there is evidence of local recurrence or metastases (except for skin Bowen's |
| | disease, or basal cell carcinoma or actinic keratoses that have been treated with no |
| | evidence of recurrence in the past 12 weeks; carcinoma in situ of the cervix or non- |
| | invasive malignant colon polyps that have been removed). |
| | 14. History or evidence of ongoing alcohol or drug abuse, which in the opinion of the |
| | investigator will prevent the patient from adhering to the protocol and completing the |
| | study. |
| | 15. Pregnant or lactating women. |
| | 16. Women of childbearing potential, defined as all women physiologically capable of |
| | becoming pregnant, unless they are using methods of contraception during the entire |
| | study or longer if required by locally approved prescribing information (e.g. in EU 20 |
| | weeks). |
| Key Design | Each study will be randomized, double-blind, placebo controlled, parallel group, multi- |
| Features | center, assessing the short and long-term efficacy, safety, and tolerability of 2 |
| | secukinumab dose regimens versus placebo in moderate to severe patients with |
| | hidradenitis suppurativa. |
| | One study will be conducted in patients using stable antibiotic drugs during the study, |
| | while the other study will not allow concomitant use of antibiotics for treating HS. |
| | Each study will consist of 3 periods: screening (up to 4 weeks), treatment period 1 (16 |
| | weeks), treatment period 2 (36 weeks). |
| | Patients will be randomized to one of 4 treatment groups: |
| | 1) Secukinumab 300 mg every 4 weeks |
| | 2) Secukinumab 300 mg every 2 weeks |
| | 3) Placebo group to secukinumab 300 mg every 4 weeks |
| | 4) Placebo group to secukinumab 300 mg every 2 weeks |
| | Both studies will have a primary endpoint at Week 16. A primary analysis will be |
| | performed once all patients have reached Week 16. |
| | At week 16 patients originally randomized to placebo will be re-randomized 1:1 to |
| | secukinumab 300 mg every 4 weeks or secukinumab 300 mg every 2 weeks. |
| | The study design and dosing schemes are shown in FIG. 3A and 3B. |

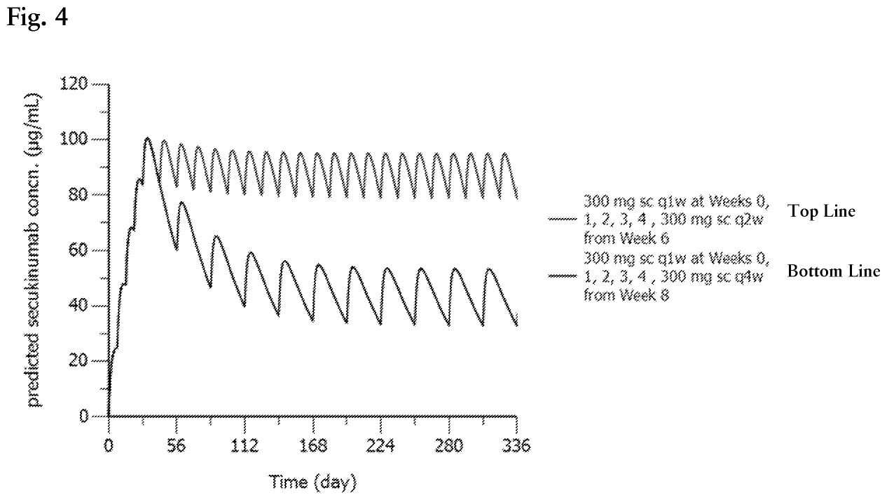

In addition to a regimen utilizing weekly induction dosing, followed by maintenance dosing every 4 weeks (Q4W), we will employ a Q2W maintenance regimen to achieve a higher exposure than with the therapeutic regimen typically used in the treatment of plaque-type psoriasis, for the following reasons:
Higher body weights are anticipated for a population of patients with HS compared to the typical psoriasis population (approximately 10 kg heavier weight for HS populations (Kimball et al (2016) N Engl J Med; 375(5):422-434), thus potentially requiring higher doses to achieve adequate exposure. Systemic exposure varies with body weight in an allometric relationship. For secukinumab clearance, the allometric exponent was estimated to be close to 1; in other words, a doubling of body weight could lead to a nearly 2-fold increase in clearance and therefore reduced serum exposure (Bruin et al (2017) J Clin Pharmacol 57(7):876-885). Therefore, evaluation of a secukinumab dosing regimen with higher exposure than resulting from the psoriasis regimen is appropriate in the HS patient population.
Higher local skin exposure than in psoriasis might be needed for this disease with deep inflammatory skin lesions.
Clinical experience with adalimumab (HUMIRA®) in HS supports a dosing regimen with higher exposure in HS than in psoriasis.
Crucially, as can be seen from the FIG. 4, we have determined that, following the same induction period during the first month, it is possible to achieve considerably higher and more consistent systemic exposure to secukinumab using a shortened maintenance dose interval (every 2 weeks) than can be reached with the 4 week maintenance interval. The dose finding strategy is further supported by FIG. 5, which shows that above ˜30 mcg/mL secukinumab trough concentration, efficacy in psoriasis patients is maximized (PASI 90˜60%).

1-46. (canceled)

47. A method of treating hidradenitis suppurativa (HS), comprising subcutaneously (SC) administering to a patient in need thereof a dose of about 300 mg of an IL-17 antibody, or an antigen-binding fragment thereof, weekly during weeks 0, 1, 2, and 3, and thereafter SC at a dose of about 300 mg every other week (every 2 weeks), beginning during week 4;
wherein the IL-17 antibody or antigen-binding fragment thereof comprises:
i) an immunoglobulin V_{H} domain comprising the amino acid sequence set forth as SEQ ID NO:8 and an immunoglobulin V_{L} domain comprising the amino acid sequence set forth as SEQ ID NO:10;
ii) an immunoglobulin V_{H} domain comprising the hypervariable regions set forth as SEQ ID NO:1, SEQ ID NO:2, and SEQ ID NO:3 and an immunoglobulin V_{L} domain comprising the hypervariable regions set forth as SEQ ID NO:4, SEQ ID NO:5 and SEQ ID NO:6; or
iii) an immunoglobulin V_{H} domain comprising the hypervariable regions set forth as SEQ ID NO:11, SEQ ID NO:12 and SEQ ID NO:13 and an immunoglobulin V_{L} domain comprising the hypervariable regions set forth as SEQ ID NO:4, SEQ ID NO:5 and SEQ ID NO:6;
and wherein the IL-17 antibody or antigen-binding fragment thereof binds to an epitope of an IL-17 homodimer having two mature IL-17 protein chains, said epitope comprising Leu74, Tyr85, His86, Met87, Asn88, Va1124, Thr125, Pro126, Ile127, Va1128, His129 on one chain and Tyr43, Tyr44, Arg46, Ala79, Asp80 on the other chain, wherein the IL-17 antibody has a K_{D} of about 100-200 pM as measured by a biosensor system, and wherein the IL-17 antibody has an in vivo half-life of about 23 to about 30 days.

48. The method according to claim 47, wherein said patient achieves a sustained response after one year of treatment, as measured by inflammatory lesion count, Hidradenitis Suppurativa Clinical Response (HiSCR), Numerical Rating Scale (NRS), modified Sartorius HS score, Hidradenitis Suppurativa-Physician Global Assessment (HS-PGA), or Dermatology Life Quality Index (DLQI).

49. The method according to claim 47, wherein said patient achieves a sustained response after one year of treatment, as measured by the simplified HiSCR (sHiSCR).

50. The method according to claim 47, wherein, prior to treatment with the IL-17 antibody or antigen-binding fragment, the patient has been previously treated with a systemic agent for HS.

51. The method according to claim 50, wherein the systemic agent is selected from the group consisting of a topical treatment, an antibiotic, an immune system suppressant, a TNF-alpha inhibitor, an IL-1 antagonist, and combinations thereof.

52. The method according to claim 47, wherein the IL-17 antibody or antigen-binding fragment is administered in combination with at least one of a TNF-alpha inhibitor, an antibiotic, an IL-1 inhibitor, or an immunosuppressant.

53. The method according to claim 47, wherein the patient has moderate to severe HS.

54. The method according to claim 47, wherein, prior to treatment with the IL-17 antibody or antigen-binding fragment, the patient has an HS-PGA score of ≥3.

55. The method according to claim 47, wherein, prior to treatment with the IL-17 antibody or antigen-binding fragment, the patient is classified under Hurley stage II or III.

56. The method according to claim 47, wherein said patient achieves a simplified HiSCR by week 16 of treatment.

57. The method according to claim 47, wherein, by week 16 of treatment, said patient achieves an NRS30 and/or a reduction of <6 as measured by the DLQI.

58. The method according to claim 47, wherein, prior to treatment with the IL-17 antibody or antigen-binding fragment thereof, the patient does not have extensive scarring (<20 fistulas) as a result of HS.

59. The method according to claim 47, wherein the patient is additionally treated with at least one topical medication and at least one antiseptic in combination with the IL-17 antibody or antigen-binding fragment thereof.

60. The method according to claim 47, wherein, as early as one week after the first dose of the IL-17 antibody or antigen-binding fragment thereof, the patient has a rapid reduction in:
a) pain, as measured by VAS or NRS, and/or
b) CRP, as measured using a standard CRP assay.

61. The method according to claim 47, wherein the patient has a reduction in modified Sartorius score and/or an improvement in DLQI by 16 weeks of treatment.

62. The method according to claim 47, wherein the IL-17 antibody or antigen-binding fragment is secukinumab.
